(19) United States
(12) Patent Application Publication
(10) Pub. No.: US 2006/0281108 A1
(43) Pub. Date: Dec. 14, 2006

(54) Compositions and methods for the analysis of degraded nucleic acids

(75) Inventors: Joseph Monforte, Kensington, CA (US); Francois Ferre, La Jolla, CA (US); Kahuku Oades, Oceanside, CA (US)

Correspondence Address: QUINE INTELLECTUAL PROPERTY LAW GROUP, P.C., P O BOX 458, ALAMEDA, CA, 94501, US

(73) Assignee: Althea Technologies, Inc., San Diego, CA (US)

(21) Appl. No.: 11/417,672

(22) Filed: May 3, 2006

Related U.S. Application Data
(60) Provisional application No. 60/677,618, filed on May 3, 2005.

(51) Int. Cl. C12Q 1/68; C12P 19/34

(52) U.S. Cl. 435/6; 435/91.2

(57) ABSTRACT

The invention relates to compositions and methods for gene expression analysis. In some embodiments, the invention provides compositions and methods for amplifying targets in a degraded nucleic acid sample. In some embodiments, the invention provides methods for determining the quality of nucleic acids (e.g., the degree of degradation) in a nucleic acid sample. The invention also provides methods for producing a gene expression profile from a degraded RNA sample.

### CROSS-REFERENCE TO RELATED APPLICATIONS

This application claims priority to and benefit of U.S. Provisional Patent Application Ser. No. 60/677,618, filed on May 3, 2005, the specification of which is hereby incorporated by reference in its entirety.

### FIELD OF THE INVENTION

The invention relates to the field of gene expression analysis. The invention provides compositions and methods for the analysis of nucleic acid samples, more specifically, methods for analyzing degraded nucleic acids and methods for determining the degree of degradation of a nucleic acid sample.

### BACKGROUND OF THE INVENTION

The analysis of gene expression has assumed a fundamental role in dissecting a wide variety of biological processes. Key to the analysis of gene expression is the collection of expressed gene products, e.g., total cellular RNA or mRNA. The integrity of the nucleic acid sample is critical in obtaining and optimizing collection of the gene expression data. Ironically, RNA samples isolated from tissues is highly susceptible to degradation, and is often unusable by current analytical methods.

Gene expression profiling can provide a key in understanding a wide variety of biological processes, e.g., oncogenesis and tumor progression. Such analysis has impacted the fields of cancer diagnosis and prognosis. That is to say, observing the changes in gene expression profiles over the course of tumor progression can provide insight into initial tumor formation, tumor progression, predicted response to various treatment regimes, and eventual outcome. Historically, clinical pathologists have collected and archived millions of cancer-specific tissue specimens over several decades. These tissue specimens are typically treated by fixation and paraffin embedding.

Although this fixation process preserves the cellular architecture, it unfortunately degrades the RNA contained in the specimen, most frequently rendering any isolated RNA ineffectual for use in common gene profiling analyses. Techniques that measure the RNA quality in these samples (or indeed in any RNA sample) for the purpose of predicting the sample usefulness in expression profiling (or any other assay or manipulation) would be a benefit to the research community. Furthermore, improved, sensitive techniques that can utilize degraded RNA samples for message amplification and expression analysis are equally useful.

Formalin-Fixed, Paraffin-Embedded (FFPE) Tissue Samples

One of the major technical problems associated with the use of formalin fixed, paraffin embedded (FFPE) tissue samples for gene expression analysis is RNA quality. Depending on a number of factors, including time between surgery and fixation, the specific method of fixation and embedding, storage time and storage conditions, the RNA within the sample will have undergone varying degrees of degradation. The more degraded the RNA, the more difficult it is to extract useful gene expression information.

If gene expression research in cancer is to progress using FFPE samples, it will be necessary to establish a series of robust, standardized methods for assessing the quality of RNA extracted from FFPE samples, and qualitative and quantitative metrics. These tool will permit quantitative RNA (or other nucleic acid) analysis even in cases where substantial nucleic acid (e.g., RNA) degradation has occurred.

Global gene expression analysis using DNA microarrays has become an essential tool in cancer research, providing detailed information about the expression responses associated with the many stages of oncogenesis, the associated clinical diagnoses and prognoses, and chemotherapy efficacy (see, e.g., van't Veer et al., (2002) “Gene Expression Profiling Predicts Clinical Outcome of Breast Cancer,” Nature 415:530-536; Vasselli et al., (2003) “Predicting Survival in Patients with Metastic Kidney Cancer Using Gene-Expression Profiling in the Primary Tumor,” PNAS 100:6958-6863; Best et al., “Molecular Differentiation of High- and Moderate-Grade Human Prostate Cancer by cDNA Microarray Analysis” Diagn. Mol. Pathol., 12:63-70; and Okutsu et al., (2002) “Prediction of Chemosensitivity for Patients with Acute Myeloid Leukemia, According to Expression Levels of 28 Genes Selected by Genome-Wide Complementary DNA Microarray Analysis,” Mol. Cancer Ther., 1:1035-1042). While many landmark studies have been undertaken, this research approach has frequently been restricted by the cost and limited availability of appropriate clinical samples, especially with respect to the performance of prospective, longitudinal studies that could provide detailed insight into long-term prognosis and survival.

An important alternative approach to ongoing prospective studies is the performance of retrospective studies that utilize, e.g., cancer tissue samples archived over the last decade. Because these samples have already been acquired and are readily available, long-term retrospective studies can be performed at a fraction of the cost of new prospective studies, and important clinical outcome associations could be identified now, rather than ten years in the future.

Clinical pathologists have been collecting and archiving millions of cancer specific tissue specimens for decades involving various protocols for fixation and paraffin embedding. A recent RAND report estimated that over 307 million tissue specimens from more than 178 million cases are stored in the United States, with additional samples being accumulated at a rate of more than 20 million per year (Eiseman and Haga, (2001) Handbook of Human Tissue Sources: A National Resource of Human Tissue Samples, Rand Report Number MR954). Tens of millions of these clinical samples are formalin-fixed, paraffin-embedded (FFPE) samples collected over the last 15 years. These samples represent an enormous potential data source for large-scale retrospective studies. The key to utilizing this data source, however, is in developing robust and validated processes that can work with the varied and often poor quality of nucleic acid that is extracted from these samples.

Protocols for fixing and embedding FFPE samples were historically developed to enable ambient, long-term storage of samples while preserving tissue structure for later microscopic analysis. These protocols were not developed with any consideration of maintaining RNA integrity for gene expression analysis. As a consequence, RNA isolated from FFPE samples is usually degraded, leading to the current situation where it is very challenging to extract gene expression information from these samples with confidence. Therefore, in order to extract useful gene expression data from FFPE samples, it is necessary to provide (a) a clear and detailed metric of the quality of each RNA sample in terms of the level of degradation of the message population, and (b) a detailed understanding of how the level of degradation impacts the accuracy of a gene expression measurement.

Analysis of gene expression levels in samples derived from FFPE tissues has been attempted with limited success using real-time PCR methods. These methods are limited to generating amplicons of typically not smaller than 70 base pairs. In a sample source comparison study by Godfrey et al., (“Quantitative mRNA Expression Analysis from Formalin-Fixed, Paraffin-Embedded Tissues Using 5′ Nuclease Quantitative Reverse Transcription-Polymerase Chain Reaction,” J. Molec. Diagnostics 2(2):84-91 (2000)) it was shown that FFPE derived RNA can accurately reflect the RNA levels in fresh unfixed tissues. These authors compared RNA extracted from fresh tissues as well as RNA extracted from FFPE samples whose pre fixation time in PBS varied and found no significant affect on the relative expression levels of the samples. This reinforces the idea that archived tissues whose prefixation times are unknown may still be a useful source of RNA for retrospective studies. This study also showed that RNA from FFPE samples was highly degraded and targeting of small amplicons, e.g. as small as 90 base pairs, decreased the Ct value of the real time reactions. Additional PCR-based studies have confirmed this observation (Specht et al., “Quantitative Gene Expression Analysis in Microdissected Archival Formalin-Fixed and Paraffin Embedded Tumor Tissue,” Amer. J. Pathology 158(2):419-429 (2001); and Cronin et al., “Measurement of gene expression in archival paraffin-embedded tissues: Development and performance of a 92-gene reverse transcriptase-polymerase chain reaction assay,” American Journal of Pathology 164(1):35-42 (2004)).

In a separate study, RNA was extracted from FFPE tissue from lymph nodes of melanoma patients and analyzed the impact of certain variables including length of time before fixation, length of fixation in addition to amplicon size. Although it was determined that the amount of total RNA extracted from FFPE samples compared to fresh tissues was markedly reduced, signal from these compromised samples could be increased as much as 100 fold by amplifying shorter amplicons (e.g. 99 base pairs) (Abrahamsen et al., “Towards Quantitative mRNA analysis in Paraffin-Embedded Tissues Using Real-Time Reverse Transcriptase-Polymerase Chain Reaction,” J. Molec. Diagnostics 5(1):34-41 (2003)).

These studies above that analyze RNA isolated from FFPE samples using real-time PCR methodologies for nucleic acid detection face limitations in their applicability due to the requirement for amplicons large enough for real-time detection by TaqMan®-style probes. For example, amplicons cannot be created in the 40-60 nucleotide range and still provide room for a TaqMan® style probe.

If suitable methods were available, FFPE samples of degraded nucleic acid (e.g., degraded RNA) can be mined for a range of RNA-based biomarkers, e.g., for multiple cancer indications, long-term disease prognoses, and to permit in-depth studies on mechanisms of oncogenesis and the impact of chemotherapy. To realize these benefits, a number of technical limitations present in the methods currently used in the art must be overcome.

RNA Quality Assessment

Historically, RNA quality has been measured by observing a few key markers. OD_{260/280} ratios provide a measure of quality in terms of contamination by protein and other cellular debris but tell nothing about the RNA integrity. RNA integrity has generally been evaluated by looking at the smear of nucleic acid using electrophoresis methods. This approach has been updated with the use of more sensitive capillary electrophoresis systems, such as the Agilent Technologies Bioanalyzer platform (e.g., the Agilent 2100). These systems provide quantitative data on the relative amounts of RNA present at a range of molecular sizes. Data is typically represented pictorially as electropherograms, see FIG. 1A, and the RNA quality is judged primarily by the relative sizes of the prominent 18S and 28S ribosomal RNA (rRNA) bands in total RNA samples. High quality RNA samples have a 28S/18S ratio near 2.0, while lower quality samples have reduced ratios and contain RNA fragments with smaller molecular weights (see FIGS. 1A and 1B). Recently, several investigators have developed alternative and more complex methods and software to score the quality of RNA samples (Auer and Lyianarachchi (2003) “Chipping away at the chip bias: RNA degradation in microarray analysis,” Nature Genetics 35(4):292-293; and see RNA Integrity Number (RIN) developed by Agilent Technologies; Schroeder et al., “The RIN: an RNA integrity number for assigning integrity values to RNA measurements,” BMC Molecular Biology 7:3 (2006), and see additional technical discussion available on the company website). These methods generally calculate a degradation factor or RIN, based on the size and number of small fragments.

Due to the fixation conditions, total RNA samples derived from FFPE samples universally appear degraded and ribosomal bands are rarely detected. The mean size of fragments generally ranges in the low hundreds (see FIG. 1C), and as a consequence, would consistently receive a poor RIN score. However, a very small fraction of FFPE samples may generate sufficient cRNA targets for microarray analysis (e.g., using Affymetrix® GeneChip® platform technology as illustrated in FIG. 12). Unfortunately, this fraction of useful FFPE samples can not be distinguished using current methods of judging RNA integrity that rely on the detection of ribosomal bands and smaller degradation fragments. Moreover, the low data yield and high cost per sample makes it impractical to run every sample on a DNA microarray platform without some prequalification of the RNA quality.

Attempts at developing additional RNA quality assays have been made. In Brooks et al., (2005; Microarray Core Services in the Functional Genomics Center of the University of Rochester Medical Center (FGC-URMC); see the Center website), it was found that traditional RNA quality evaluations did not always identify samples that performed poorly in microarray hybridizations. Brooks et al. developed an assay that evaluates RNA samples after they have been reverse transcribed into cDNA. Primer sets are designed from three regions (5′, middle or 3′) from a set of three transcripts known to be present in the input RNA at multiple concentrations (low, medium and high). The cDNA samples are individually assayed with the nine primer sets using real-time PCR. The presence or absence of an amplified product (but not the quantitative Ct values) is recorded for each primer set. A QC score is calculated for each sample based on the number of primer sets that generated amplified products and whether all genes were detected. By interrogating genes expressed at different levels and primers from different gene locations, this type of cDNA metric has been used to pre-qualify a sample for inclusion in expression analysis. Although this method has some advantages over electrophoretic analysis, the approach only looks at a very limited set of data (approximately 9 data points) and cannot generally work with degraded, FFPE-sample-derived RNA. Nor will the assay incorporate any information with relation to amplification efficiency as a function of amplicon size.

There is a need in the art for novel, improved approaches to assessing the quality of RNA for use in both microarrays (e.g., for global expression profiling) and PCR-based studies. There is a need in the art for PCR methods for assessing the integrity of a sampling of RNA transcripts; e.g., where the methods utilize highly multiplexed PCR amplification. There is a need in the art for innovative PCR methods for the assessment of nucleic acid (e.g., RNA) quality and suitability for use in, , e.g., microarray analysis. These techniques will enable data to be routinely extracted from the large archive of FFPE samples. These FFPE samples can be mined for a range of RNA-based biomarkers e.g., for multiple cancer indications, long-term disease prognoses, and in-depth studies on mechanisms of oncogenesis, the impact of chemotherapy and other factors.

There continues to be a strong need for new methods that generate a degradation metric that can distinguish the highly degraded RNA samples from the partially degraded RNA samples. Ideally, the samples having a degradation value that falls below a given threshold should be excluded from subsequent gene expression studies, and samples that have a degradation value above a given threshold should be included in subsequent gene expression studies. Furthermore, there is a need in the art for degradation metric assays to be more than just a determination of whether or not a sample can generate microarray data. Preferably, the quality metric should also ideally provide information on what the potential level of quality is, with relation to accuracy and precision, that will yield from a given sample.

The present invention provides compositions and methods that meet these needs in the art, and provide other advantages, as described in the present specification.

### SUMMARY OF THE INVENTION

The present invention provides new PCR-based methods for amplifying degraded nucleic acids, accurately and directly measuring the level of degradation within a nucleic acid population (e.g., mRNA or genomic DNA), provides a gene-level metric of nucleic acid (e.g., RNA) quality, and provides methods for producing gene expression profiles using degraded RNA starting material. This invention provides new methods which are improvements over the art and provides a system for assessing the quality of nucleic acid, and furthermore, provides methods that permit the use of degraded formalin-fixed, parafin-embedded (FFPE) tissue samples for gene expression analysis.

In some aspects, the invention provides methods for amplifying members of a population of degraded nucleic acids in a sample, where the method comprises the steps of:

a) providing a sample comprising said population of degraded nucleic acids;

b) providing target primer pairs, where (i) each target primer pair comprises a forward target primer and a reverse target primer; (ii) said forward and reverse target primers each comprise (A) a target-specific nucleotide sequence that is complementary to a nucleotide subsequence of at least one member of the population of degraded nucleic acids, and (B) at least one universal priming sequence, wherein the universal priming sequence is 5′ relative to the target-specific sequence; and

c) annealing the target primer pairs to their cognate degraded nucleic acid;

d) enzymatically producing a plurality of products corresponding to subsequences of the cognate degraded nucleic acids;

e) enzymatically amplifying the plurality of products using at least one universal primer to produce a plurality of target amplicons, wherein the universal primer comprises nucleotide sequence that is complementary to the universal priming sequence, thereby amplifying members of a population of degraded nucleic acids.

In some embodiments, the degraded nucleic acids are DNA, and the sample has a mean size of not more than 1,000 nucleotides. In other embodiments, the nucleic acids are RNA, and the population a mean size of not more than about, alternatively, 600 nucleotides, 450 nucleotides. about or less than 300 nucleotides.

The number of amplified population members is not limited. For example, the number of members that are amplified can be between about 2 and about 100 members, between about 10 and about 40 members. In some aspects, the population of degraded nucleic acids is expressed gene nucleotide sequences (e.g., mRNA molecules). In some aspects, the expressed gene nucleotide sequence is a constitutively-expressed reference gene. In some aspects, the method entails comparing a level of expression of at least one expressed gene sequence to a level of expression of at least one other expressed gene sequence. Alternatively, the expressed gene nucleotide sequence can be a tissue-specific gene sequence.

In these methods, the population of degraded nucleic acids can be RNA or DNA. Where the nucleic acid is RNA, the step of producing a plurality of products can use reverse transcription. When degraded mammalian RNA is used, the quantitative ratio of 28S RNA to 18S RNA is not more than 2.0:1, or alternatively, not more than 1.8:1. In some aspects, the sample comprises total cellular RNA, or alternatively, polyadenylated RNA. In some aspects, the sample is derived from a tissue sample that has undergone fixation, such as a paraffin-embedded tissue sample.

In some embodiments, at least one member in the plurality of products produced by the method has not more than 200 base pairs of nucleotide sequence corresponding to the degraded nucleic acid target, or alternatively, not more than 100 base pairs, not more than 80 base pairs, or not more than 60 base pairs. In the methods of teh invention, the enzymatically producing a plurality of products can use enzymatic nucleic acid polymerization by the polymerase chain reaction (PCR), for example, multiplex PCR. In some aspects, the multiplex PCR uses between about 2 and about 100 target primer pairs, or alternatively, between about 10 and about 40 target primer pairs.

In some embodiments of the methods, at least one target primer can further contains at least one spacer nucleotide between the target-specific sequence and the universal priming sequence. In these methods, the concentration of each target primer in the target primer pair can be less than the concentration of the at least one universal primer. The ratio of the concentration of each target primer pair to the concentration of the universal primer can be between about 1:2 and 1:1000, or alternatively, between about 1:10 and 1:100.

In these methods, the enzymatically amplifying the plurality of products can use enzymatic nucleic acid polymerization by the polymerase chain reaction (PCR). In these methods, the length of one target amplicon can be different than the length of at least a second target amplicon. In other aspects, the 3′ end of the forward primer is not more than 20 base pairs from the 3′ end of the reverse primer when the target primers are hybridized to a cognate nucleic acid target. At least one target amplicon can comprises a label, and furthermore, a plurality of the target amplicons can each comprise a different label.

In these methods, the plurality of target amplicons can be detected, for example, by capillary electrophoresis analysis, or alternatively, by hybridization analysis, e.g., an array hybridization or a bead system hybridization.

In other aspects, the invention provides a method for determining nucleic acid quality in a nucleic acid sample (i.e., a nucleic acid quality metric). This method has the steps:

a) providing a nucleic acid sample;

b) providing at least two target primer pairs, where: (i) each target primer pair comprises a forward target primer and a reverse target primer; (ii) the forward and reverse target primers each comprise a target-specific nucleotide sequence that is complementary to a subsequence of at least one nucleic acid in the sample; and

c) annealing the target primer pairs to their cognate nucleic acid;

d) enzymatically producing at least two products corresponding to nucleotide subsequences of the cognate nucleic acid, where each nucleotide subsequence is a different length;

e) enzymatically amplifying the products to produce at least two target amplicons;

f) quantitating the at least two target amplicons; and

g) comparing quantities of the target amplicons, thereby determining nucleic acid quality in the sample.

In some aspects of these methods, the at least two target primer pairs each anneal to the same nucleic acid. The at least two products can comprise overlapping or non-overlapping nucleotide subsequences. In some embodiments of these methods, the forward and reverse target primers each further comprise at least one universal priming sequence, where the universal priming sequence is 5′ relative to the target-specific sequence; and where enzymatically amplifying the products comprises incorporating at least one universal primer to produce at least two target amplicons, where the universal primer comprises nucleotide sequence that is complementary to the universal priming sequence. Further, the at least one target primer can further contain at least one spacer nucleotide between the target-specific sequence and the universal priming sequence. In some aspects, the at least two target primer pairs anneal to different nucleic acids.

In these methods, where each product comprises nucleotide sequence corresponding to a cognate nucleic acid, where the nucleotide sequence has a size range selected from about (i) 40-60 base pairs, inclusive; (ii) 100-120 base pairs, inclusive; and (iii) 180-200 base pairs, inclusive, where the nucleotide sequence size range for one product is different than the nucleotide sequence size range of any other product. In some aspects, only two target primer pairs are used, or alternatively, three target primer pairs are used.

In some embodiments of these methods, each product comprises nucleotide sequence corresponding to a cognate nucleic acid, where the nucleotide sequence has a size range selected from about (i) 40-60 base pairs, inclusive; (ii) 100-120 base pairs, inclusive; and (iii) 180-200 base pairs, inclusive, where the nucleotide sequence size range for one product is different than the nucleotide sequence size range of the remaining two products.

In some embodiments, the comparing quantities of the target amplicons comprises comparing relative molar concentrations of the target amplicons. In some aspects, the relative molar concentration of one target amplicon is less than the relative molar concentration of at least a second target amplicon, thereby indicating degraded nucleic acid.

The invention provides methods for producing a gene expression profile from a degraded RNA sample, the method comprising the steps:

a) providing a sample comprising degraded RNA, where the RNA corresponds to expressed genes;

b) providing a plurality of target primer pairs, where: (i) each target primer pair comprises a forward target primer and a reverse target primer; (ii) the forward and reverse target primers each comprise (A) a target-specific nucleotide sequence that is complementary to a nucleotide subsequence of at least one degraded RNA in the sample, and (B) at least one universal priming sequence, where the universal priming sequence is 5′ relative to the target-specific sequence; and

c) annealing the target primer pairs to their cognate degraded RNA;

d) enzymatically producing a plurality of products corresponding to subsequences of the cognate degraded RNA;

e) enzymatically amplifying the plurality of products using at least one universal primer to produce a plurality of target amplicons, where the universal primer comprises nucleotide sequence that is complementary to the universal priming sequence, thereby producing a gene expression profile from a degraded RNA sample.

In these methods, the plurality of target primer pairs can comprise between about between about 2 and about 100 target primer pairs, or alternatively, between about 10 and about 40 target primer pairs. In some aspects, at least one target amplicon comprises a label. In other aspects, a plurality of the target amplicons each comprise a different label. In the methods, the plurality of target amplicons can be detected, for example, by capillary electrophoresis or by hybridization analysis such as an array hybridization or a bead system hybridization.

In some embodiments, teh invention provides a kit for analyzing a sample, the sample comprising degraded nucleic acid, the kit comprising:

a) a plurality of target primer pairs, where: (i) each target primer pair comprises a forward target primer and a reverse target primer; (ii) the forward and reverse target primers each comprise (A) a target-specific nucleotide sequence that is complementary to a nucleotide subsequence of at least one degraded nucleic acid in the sample, and (B) at least one universal priming sequence, where the universal priming sequence is 5′ relative to the target-specific sequence; and (iii) each primer in a target primer pair comprises a 3′ end, and where the 3′ end of the forward primer is not more than 20 base pairs from the 3′ end of the reverse primer when the target primers are hybridized to a cognate nucleic acid target; and (b) instructions for analyzing a sample comprising degraded nucleic acids.

Definitions

Before describing the invention in detail, it is to be understood that this invention is not limited to particular biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes combinations of two or more cells; reference to “a polynucleotide” includes, as a practical matter, many copies of that polynucleotide.

Unless defined herein and below in the reminder of the specification, all technical and scientific terms used herein have the same meaning as commonly understood by one of ordinary skill in the art to which the invention pertains. In describing and claiming the present invention, the following terminology will be used in accordance with the definitions set out below.

Base: As used herein, the term “base” refers to any nitrogen-containing heterocyclic moiety capable of forming Watson-Crick type hydrogen bonds in pairing with a complementary base or base analog. A large number of natural and synthetic (non-natural, or unnatural) bases, base analogs arid base derivatives are known. Examples of bases include purines and pyrimidines, and modified forms thereof. The naturally occurring bases include, but are not limited to, adenine (A), guanine (G), cytosine (C), uracil (U) and thymine (T). As used herein, it is not intended that the invention be limited to naturally occurring bases, as a large number of unnatural (non-naturally occurring) bases and their respective unnatural nucleotides that find use with the invention are known to one of skill in the art. Examples of such unnatural bases are given below.

Nucleoside: The term “nucleoside” refers to a compound consisting of a base linked to the C-1′ carbon of a sugar, for example, ribose or deoxyribose.

Nucleotide: The term “nucleotide” refers generally to a phosphate ester of a nucleoside, as a monomer unit or within a polynucleotide. “Nucleotide 5′-triphosphate” refers to a nucleotide with a triphosphate ester group attached to the sugar 5′-carbon position, and are sometimes denoted as “NTP”, or “dNTP” and “ddNTP.” A modified nucleotide is any nucleotide (e.g., ATP, TTP, GTP or CTP) that has been chemically modified, typically by modification of the base moiety. Modified nucleotides include, for example but not limited to, methylcytosine, 6-mercaptopurine, 5-fluorouracil, 5-iodo-2′-deoxyuridine and 6-thioguanine. As used herein, the term “nucleotide analog” refers to any nucleotide that is non-naturally occurring.

Polynucleotide or nucleic acid: The terms “nucleic acid,” “nucleic acid sequence,” “polynucleotide,” “polynucleotide sequence,” “oligonucleotide,” “oligomer,” “oligo” or the like, as used herein, refer to a polymer of monomer subunits that can be corresponded to a sequence of nucleotide bases, e.g., a DNA (e.g., cDNA), RNA (e.g., mRNA, rRNA, tRNA, small nuclear RNAs), peptide nucleic acid (PNA), RNA/DNA copolymers, any analogues thereof, or the like. A polynucleotide can be single- or double-stranded, and can be complementary to the sense or antisense strand of a gene sequence. A polynucleotide can hybridize with a complementary portion of a target polynucleotide to form a duplex, which can be a homoduplex or a heteroduplex. The length of a polynucleotide is not limited in any respect. Linkages between nucleotides can be internucleotide-type phosphodiester linkages, or any other type of linkage. A “polynucleotide sequence” refers to the sequence of nucleotide monomers along the polymer. A “polynucleotide” is not limited to any particular length or range of nucleotide sequence, as the term “polynucleotide” encompasses polymeric forms of nucleotides of any length. A polynucleotide can be produced by biological means (e.g., enzymatically by a nucleic acid polymerase enzyme, for example, by a thermostable DNA polymerase during a PCR reaction), or synthesized using an enzyme-free system. A polynucleotide can be enzymatically extendable or enzymatically non-extendable. Unless otherwise indicated, a particular polynucleotide sequence of the invention optionally encompasses complementary sequences, in addition to the sequence explicitly indicated. Nucleic acid can be obtained from any source, for example, a cellular extract, genomic or extragenomic DNA, viral RNA or DNA, or artificially/chemically synthesized molecules. Unless otherwise indicated, a particular polynucleotide sequence encompasses complementary sequences in addition to the sequence explicitly indicated. Furthermore, any nucleic acid can comprise a nucleotide subsequence that comprises any portion of the nucleic acid, where the subsequence is shorter than the original nucleic acid by at least one nucleotide.

Polynucleotides that are formed by 3′-5′ phosphodiester linkages are said to have 5′-ends and 3′-ends because the nucleotide monomers that are reacted to make the polynucleotide are joined in such a manner that the 5′ phosphate of one mononucleotide pentose ring is attached to the 3′ oxygen (hydroxyl) of its neighbor in one direction via the phosphodiester linkage. Thus, the 5′-end of a polynucleotide molecule has a free phosphate group or a hydroxyl at the 5′ position of the pentose ring of the nucleotide, while the 3′ end of the polynucleotide molecule has a free phosphate or hydroxyl group at the 3′ position of the pentose ring. Within a polynucleotide molecule, a position or sequence that is oriented 5′ relative to another position or sequence is said to be located “upstream,” while a position that is 3′ to another position is said to be “downstream.” This terminology reflects the fact that polymerases proceed and extend a polynucleotide chain in a 5′ to 3′ fashion along the template strand. Unless denoted otherwise, whenever a polynucleotide sequence is represented, it will be understood that the nucleotides are in 5′ to 3′ orientation from left to right.

As used herein, it is not intended that the term “polynucleotides” be limited to naturally occurring polynucleotides sequences or polynucleotide structures, naturally occurring backbones or naturally occurring internucleotide linkages. One familiar with the art knows well the wide variety of polynucleotide analogues, unnatural nucleotides, non-natural phosphodiester bond linkages and internucleotide analogs that find use with the invention. Non-limiting examples of such unnatural structures include non-ribose sugar backbones, 3′-5′ and 2′-5′ phosphodiester linkages, internucleotide inverted linkages (e.g., 3′-3′ and 5′-5′), branched structures, and internucleotide analogs (e.g., peptide nucleic acids (PNAs), locked nucleic acids (LNAs), C₁-C₄ alkylphosphonate linkages such as methylphosphonate, phosphoramidate, C₁-C₆ alkyl-phosphotriester, phosphorothioate and phosphorodithioate internucleotide linkages. Furthermore, a polynucleotide can be composed entirely of a single type of monomeric subunit and one type of linkage, or can be composed of mixtures or combinations of different types of subunits and different types of linkages (a polynucleotide can be a chimeric molecule). As used herein, a polynucleotide analog retains the essential nature of natural polynucleotides in that they hybridize to a single-stranded nucleic acid target in a manner similar to naturally occurring polynucleotides.

RNA: The term “RNA,” an acronym for ribonucleic acid, refers to any polymer of ribonucleotides. The term “RNA” can refer to polymers comprising natural, unnatural or modified ribonucleotides, or any combinations thereof (i.e., chimeric RNA molecules). The term “RNA” includes all biological forms of RNA, including for example, mRNA (typically polyA RNA), rRNA (ribosomal RNA), tRNA (transfer RNA), and small nuclear RNAs, as well as non-naturally occurring forms of RNA, including cRNA, antisense RNA, and any type of artificial (e.g., recombinant) transcript not endogenous to a cellular system. The term “total cellular RNA” generally refers to the RNA that is isolated from cells using isolation techniques that do not discriminate between the different types of RNA in the cell. Thus, a total cellular RNA sample will contain mRNA, tRNA, rRNA and other types of RNA. The term polyadenylated RNA refers to RNA that has a poly-A tail, generally used interchangeably with “mRNA.”

The term RNA also encompasses RNA molecules that comprise non-natural ribonucleotide analogues, such as 2-O-methylated ribonucleotides. RNA can be produced by any method, including by enzymatic synthesis or by artificial (chemical) synthesis. Enzymatic synthesis can include cell-free in vitro transcription systems and cellular systems, e.g., in a prokaryotic cell or in a eukaryotic cell.

cDNA: The term “cDNA” refers to “complementary” or “copy” DNA. Generally cDNA is synthesized by an RNA-dependent DNA polymerase having reverse transcriptase activity (e.g., a nucleic acid polymerase that uses an RNA template to generate a complementary DNA molecule) using any type of RNA molecule (e.g., typically mRNA) as a template. Alternatively, the cDNA can be obtained by directed chemical syntheses.

Degraded—As used herein, the term “degraded” as it applies to a nucleic acid molecule refers to the state of the molecule where the length of the molecule is shorter than the predicted full-length of that molecule in its in vivo environment in a living cell. Alternatively, the term degraded can refer to the state of a single nucleic acid molecule where the length of the molecule is shorter than the experimentally observed length of that molecule following isolation of a sample using techniques known in the art to preserve molecular integrity.

As used herein, a discussion of the singular form “a nucleic acid molecule” or the like includes many copies of that molecule. As a practical matter, most techniques for visualizing, detecting, isolating or otherwise manipulating a nucleic acid molecule apply to a plurality of that molecule. For example, a single band on a Northern blot does not indicate the size of a single RNA molecule, but rather, it reflects the size of the vast majority of transcripts for that particular RNA species.

Application of the term “degraded” can be illustrated by the following example. A particular expressed gene X is predicted (from its cloned cDNA and/or genomic sequences) to encode a 2,000 base pair mRNA. In one aspect, any gene X mRNA in any sample that is shorter than 2,000 base pairs is a degraded mRNA.

In another aspect, nucleic acid samples (for example polyA-RNA samples) can be isolated from cells using methods for the preservation of RNA quality (e.g., methods that use DEPC, RNase or DNase inhibitors, low shear forces, etc). When gene X mRNA expression is analyzed in these RNA samples, for example by Northern blot, a predominant band of approximately 1,950 base pairs in length is observed. In one aspect, any gene X mRNA that is shorter than 1,950 base pairs in length is a degraded gene X mRNA.

In another aspect, the determination of whether a sample comprising mammalian total RNA is degraded or undegraded is made by observing the quantitation ratio of 28S ribosomal RNA to 18S ribosomal RNA. This technique is well established and widely used in the art. In some embodiments, a 28S:18S ratio of 2.0:1 is a suitable benchmark to define an undegraded RNA sample, where any ratio less than 2.0:1 is considered degraded. In other embodiments, especially where the RNA sample is derived from a tissue sample from an organism, a ratio of 1.8:1 is considered a suitable benchmark to define an undegraded RNA sample, and any sample with a 28S:18S ratio less than 1.8:1 is considered degraded.

In some embodiments, a sample of total cellular RNA or polyA-RNA is degraded if the sample has a mean nucleic acid size of approximately 600 nucleotides or smaller. Alternatively, a sample of total cellular RNA or polyA-RNA is degraded if the sample has a mean nucleic acid size of approximately 450 nucleotides or smaller. Alternatively still, a sample of total cellular RNA or polyA-RNA is degraded if the sample has a mean nucleic acid size of approximately 300 nucleotides or smaller. The mean size of the nucleic acids in the sample can be determined by any suitable method, for example, by agarose gel electrophoresis or polyacrylamide gel electrophoresis in conjunction with a suitable staining/visualization protocol. Alternatively, the mean size of the nucleic acids in the sample can be determined by chromatography, various size fractionating micro and nanofluidic methods, or microscopic and mass spectrometric methods as known in the art. Note that RNA degradation can be determined by observation of the RNA directly or cDNA and cRNA products as derived from the potentially degraded RNA.

In other aspects, samples used in the present invention are degraded genomic DNA samples. Various standards are known in the art for assessing the state of genomic DNA degradation. These standards and techniques are in some cases different than the criteria used to judge degraded RNA. In is broadest sense in vivo, any piece of DNA shorter than the length of an intact full-length chromosome can be considered a degraded DNA molecule. However, as a practical matter, genomic DNA must be fragmented to some degree to permit laboratory manipulation. This deliberate fragmentation can occur by any suitable means, including but not limited to mechanical shearing or enzymatic or chemical digestion. The state of genomic DNA degradation can be determined by visualizing a mean fragment size following fragmentation. For example, a sample of genomic DNA having a mean fragment size of about 10,000 nucleotides or more can be considered intact for purposes of most laboratory analysis. In other embodiments, a sample of genomic DNA having a mean fragment size of about 5,000 nucleotides or more can be considered intact (i.e., undegraded). In other embodiments, a sample of genomic DNA having a mean fragment size of about 2,000 nucleotides or more can be considered intact (i.e., undegraded). Smaller mean fragment sizes, termed herein “degraded” can frequently be observed, whether deliberately generated (e.g., by specific enzymatic digestion or shearing) or unintentional (for example, by improper sample storage, tissue treatments such as fixation processes, or by cellular mechanisms such as apoptosis or necrosis). In some embodiments, a degraded genomic DNA sample can be defined as a DNA sample having a mean fragment size of about 1,000 nucleotides or smaller. Electrophoresis (e.g., agarose gel electrophoresis) and staining can be used to measure the size of DNA and extent of degradation

Amplification: As used herein, the terms “amplification,” “amplifying” and the like refer generally to any process that results in an increase in the copy number of a molecule or set of related molecules. As it applies to polynucleotide molecules, amplification means the production of multiple copies of a polynucleotide molecule, or a portion of a polynucleotide molecule, typically starting from a small amount of a polynucleotide (e.g., an mRNA), where the amplified material (e.g., a cDNA) is typically detectable. Amplification of polynucleotides encompasses a variety of chemical and enzymatic processes. The generation of multiple DNA copies from one or a few copies of a template DNA molecule during a polymerase chain reaction (PCR), a strand displacement amplification (SDA) reaction, a transcription mediated amplification (TMA) reaction, a nucleic acid sequence-based amplification (NASBA) reaction, or a ligase chain reaction (LCR) are forms of amplification. Amplification is not limited to the strict duplication of the starting molecule. For example, the generation of multiple cDNA molecules from a limited amount of viral RNA in a sample using RT-PCR is a form of amplification. Furthermore, the generation of multiple RNA molecules from a single DNA molecule during the process of transcription is also a form of amplification.

In some embodiments, amplification is optionally followed by additional steps, for example, but not limited to, labeling, sequencing, purification, isolation, hybridization, size resolution, expression, detecting and/or cloning.

Polymerase Chain Reaction: As used herein, the term “polymerase chain reaction” (PCR) refers to a method for amplification well known in the art for increasing the concentration of a segment of a target polynucleotide in a sample, where the sample can be a single polynucleotide species, or multiple polynucleotides. Generally, the PCR process consists of introducing a molar excess of two or more extendable oligonucleotide primers to a reaction mixture comprising the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers. Reverse transcriptase PCR (RT-PCR) is a PCR reaction that uses RNA template and a reverse transcriptase, or an enzyme having reverse transcriptase activity, to first generate a single stranded DNA molecule prior to the multiple cycles of DNA-dependent DNA polymerase primer elongation. Methods for a wide variety of PCR applications are widely known in the art, and described in many sources, for example, Ausubel et al. (eds.), Current Protocols in Molecular Biology, Section 15, John Wiley & Sons, Inc., New York (1994).

Multiplex PCR: The term “multiplex PCR” or “multiplex reaction” refer to PCR reactions that produce more than one amplified product in a single reaction mixture, typically by the inclusion of more than two primers in a single reaction. The term “multiplex amplification” refers to a plurality of amplification reactions conducted simultaneously in a single reaction mixture. In the context of the present invention, the term “simultaneously” means that more than one reaction (e.g., a plurality of hybridization reactions) occur at substantially the same time. For example, reagents to be hybridized, such as more than two amplification primers, are contacted at the same time and/or in the same solution with target nucleic acids.

Target: As used herein, “target”, “target polynucleotide”, “target sequence” and the like refer to a specific polynucleotide sequence that is the subject of hybridization with a complementary polynucleotide, e.g., a DNA polymerase primer. The hybridization complex formed as a result of the annealing of a polynucleotide with its target is termed a “target hybridization complex.” The hybridization complex can form in solution (and is therefore soluble), or one or more component of the hybridization complex can be affixed to a solid phase (e.g., to a dot blot, affixed to a bead system to facilitate removal or isolation of target hybridization complexes, or in a microarray). The structure of the target sequence is not limited, and can be composed of DNA, RNA, analogs thereof, or combinations thereof, and can be single-stranded or double-stranded. A target polynucleotide can be derived from any source, including, for example, any living or once living organism, including but not limited to prokaryote, eukaryote, plant, animal, and virus, as well as synthetic and/or recombinant target sequences. Ins some aspects, the presence, absence or abundance of the “target” is to be determined. Alternatively, a target can be amplified.

Template: Generally, the term “template” refers to any nucleic acid polymer that can serve as a sequence that can be copied into a complementary sequence by the action of, for example, a polymerase enzyme. In some aspects, the target polynucleotide in a hybridization complex serves as a “template,” where an extendable polynucleotide primer binds to the template and initiates nucleotide polymerization using the base sequence of the template as a pattern for the synthesis of a complementary polynucleotide.

Primer or Target Primer or Target-Specific Primer: As used herein, the terms “primer” or “target primer” or the like refer to a 3′-enzymatically extendable oligonucleotide, generally with a defined sequence that is designed to hybridize in an antiparallel manner with a complementary (or partially complementary) primer-specific portion of a target sequence; that is to say, a primer has at least one cognate target nucleic acid. Further, a primer can initiate the polymerization of nucleotides in a template-dependent manner to yield a polynucleotide that is complementary to the target polynucleotide. The extension of a primer annealed to a target uses a suitable DNA or RNA polymerase in suitable reaction conditions. One of skill in the art knows well that polymerization reaction conditions and reagents are well established in the art, and are described in a variety of sources.

A primer nucleic acid does not need to have 100% complementarity with its template subsequence for primer elongation to occur; primers with less than 100% complementarity can be sufficient for hybridization and polymerase elongation to occur. Optionally, a primer nucleic acid can be labeled, if desired. The label used on a primer can be any suitable label, and can be detected by, for example, by spectroscopic, photochemical, biochemical, immunochemical, chemical, or other detection means.

Universal Primer: The term “universal primer” refers to a primer comprising a universal sequence that is able to hybridize to all, or essentially all, potential target sequences in a multiplexed reaction. The term “semi-universal primer” refers to a primer that is capable of hybridizing with more than one (e.g., a subset), but not all, of the potential target sequences in a multiplexed reaction. The terms “universal sequence,” “universal priming sequence” or “universal primer sequence” or the like refer to a sequence contained in a plurality of primers, where the universal priming sequence that is found in a target is complementary to a universal primer.

Primer Pair or Amplification Primer Pair: As used herein, the expression “primer pair” or “amplification primer pair” refers to a set of two primers that are generally in molar excess relative to their target polynucleotide sequence, and together prime template-dependent enzymatic DNA synthesis and amplification of the target sequence to yield a double-stranded amplicon. A primer pair is sometimes said to consist of a “forward primer” and a “reverse primer,” (or left primer and right primer) indicating that they are initiating nucleic acid polymerization in opposing directions from different strands of the target duplex.

Amplicon: As used herein, the term “amplicon” refers to a polynucleotide molecule (or collectively the plurality of molecules) produced following the amplification of a particular target nucleic acid. The amplification method used to generate the amplicon can be any suitable method, most typically, for example, by using a PCR methodology. An amplicon is typically, but not exclusively, a DNA amplicon. An amplicon can be single-stranded or double-stranded, or in a mixture thereof in any concentration ratio.

Real-time PCR: As used herein, the expression “real-time PCR” refers to the detection of, and typically the quantitation thereof, of a specific amplicon or amplicons, as the amplicon(s) is/are being produced by PCR, without the need for a detection or quantitation step following the completion of the amplification. A common method for real-time detection of amplicon accumulation is by a 5′-nuclease assay, also termed a fluorogenic 5′-nuclease assay, e.g., a TaqMan® analysis; see, Holland et al., Proc. Natl. Acad. Sci. USA 88:7276-7280 (1991); and Heid et al., Genome Research 6:986-994 (1996). In the TaqMan® PCR procedure, two oligonucleotide primers are used to generate an amplicon specific to the PCR reaction. A third oligonucleotide (the TaqMan® probe) is designed to hybridize with a nucleotide sequence in the amplicon located between the two PCR primers. The probe may have a structure that is non-extendible by the DNA polymerase used in the PCR reaction, and is typically (but not necessarily) colabeled with a fluorescent reporter dye and a quencher moiety in close proximity to one another. The emission from the reporter dye is quenched by the quenching moiety when the fluor and quencher are in close proximity, as they are on the probe. In some cases, the probe may be labeled with only a fluorescent reporter dye or another detectable moiety.

The TaqMan® PCR reaction uses a thermostable DNA-dependent DNA polymerase that possesses a 5′-3′ nuclease activity. During the PCR amplification reaction, the 5′-3′ nuclease activity of the DNA polymerase cleaves the labeled probe that is hybridized to the amplicon in a template-dependent manner. The resultant probe fragments dissociate from the primer/template complex, and the reporter dye is then free from the quenching effect of the quencher moiety. Approximately one molecule of reporter dye is liberated for each new amplicon molecule synthesized, and detection of the unquenched reporter dye provides the basis for quantitative interpretation of the data, such that the amount of released fluorescent reporter dye is directly proportional to the amount of amplicon template.

One measure of the TaqMan® assay data is typically expressed as the threshold cycle (C_{T}), where the PCR cycle number when the fluorescence signal is first recorded as statistically significant, or where the fluorescence signal is above some other arbitrary level (e.g., the arbitrary fluorescence level, or AFL), is the threshold cycle (C_{T}).

Protocols and reagents for 5′-nuclease assays are well known to one of skill in the art, and are described in various sources. For example, 5′-nuclease reactions and probes are described in U.S. Pat. No. 6,214,979, entitled “HOMOGENEOUS ASSAY SYSTEM,” issued Apr. 10, 2001 to Gelfand et al.; U.S. Pat. No. 5,804,375, entitled “REACTION MIXTURES FOR DETECTION OF TARGET NUCLEIC ACIDS,” issued Sep. 8, 1998 to Gelfand et al.; U.S. Pat. No. 5,487,972, entitled “NUCLEIC ACID DETECTION BY THE 5′-3′ EXONUCLEASE ACTIVITY OF POLYMERASES ACTING ON ADJACENTLY HYBRIDIZED OLIGONUCLEOTIDES,” issued Jan. 30, 1996 to Gelfand et al.; and U.S. Pat. No. 5,210,015, entitled “HOMOGENEOUS ASSAY SYSTEM USING THE NUCLEASE ACTIVITY OF A NUCLEIC ACID POLYMERASE,” issued May 11, 1993 to Gelfand et al., all of which are incorporated by reference. A variety of variations in for real-time PCR methodologies are also well known.

Complementary. The terms “complementary” or “complementarity” refer to nucleic acid sequences capable of base-pairing according to the standard Watson-Crick complementary rules, or being capable of hybridizing to a particular nucleic acid segment under relatively stringent conditions. Optionally, nucleic acid polymers are optionally complementary across only portions of their entire sequences. As used herein, the terms “complementary” or are used in reference to antiparallel strands of polynucleotides related by the Watson-Crick (and optionally Hoogsteen-type) base-pairing rules. For example, the sequence 5′-AGTTC-3′ is complementary to the sequence 5′-GAACT-3′. The terms “completely complementary” or “100% complementary” and the like refer to complementary sequences that have perfect Watson-Crick pairing of bases between the antiparallel strands (no mismatches in the polynucleotide duplex). The terms “partial complementarity,” “partially complementary,” “incomplete complementarity” or “incompletely complementary” and the like refer to any alignment of bases between antiparallel polynucleotide strands that is less than 100% perfect (e.g., there exists at least one mismatch in the polynucleotide duplex). Furthermore, two sequences are said to be complementary over a portion of their length if there exist one or more mismatch, gap or insertion in their alignment. A single-stranded nucleic acid “complement” refers a single nucleic acid strand that is complementary or partially complementary to a given single nucleic acid strand.

Furthermore, a “complement” of a target polynucleotide refers to a polynucleotide that can combine (e.g., hybridize) in an antiparallel association with at least a portion of the target polynucleotide. The antiparallel association can be intramolecular, e.g., in the form of a hairpin loop within a nucleic acid molecule, or intermolecular, such as when two or more single-stranded nucleic acid molecules hybridize with one another.

Hybridize or Anneal: As used herein, two nucleic acids are said to “hybridize” or “anneal” or “bind” when they associate with one another, typically in solution, typically by a base-pairing phenomenon between antiparallel nucleic acid molecules that results in formation of a duplex or other higher-ordered structure, typically termed a hybridization complex. The ability of two regions of complementarity to hybridize is dependent on the length and continuity of the complementary regions, and the stringency of hybridization conditions. In describing hybridization between any two nucleic acids (e.g., between an array probe and an amplified RNA target such as a cDNA), sometimes the hybridization encompasses only a portion of the target or probe. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference. Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides. Both Hames and Higgins 1 and 2 are incorporated by reference.

The primary interaction between the antiparallel polynucleotide molecules is typically base specific, e.g., A/T and G/C, by Watson/Crick and/or Hoogsteen-type hydrogen bonding. It is not a requirement that two polynucleotides have 100% complementarity over their full length to achieve hybridization. In some aspects, a hybridization complex can form from intermolecular interactions, or alternatively, can form from intramolecular interactions.

Specifically hybridize: As used herein, the phrases “specifically hybridize,” “specific hybridization” and the like refer to hybridization resulting in a complex where the annealing pair show complementarity, and preferentially bind to each other to the exclusion of other potential binding partners in the hybridization reaction. It is noted that the term “specifically hybridize” does not require that a resulting hybridization complex have 100% complementarity; hybridization complexes that have mismatches can also specifically hybridize and form a hybridization complex.

Stringent hybridization: As used herein, “stringent hybridization” conditions or “stringent conditions” in the context of nucleic acid hybridization are sequence dependent, and are different under different environmental parameters. An extensive guide to hybridization of nucleic acids is found in Tijssen (1993), supra. Generally, “highly stringent” hybridization and wash conditions are selected to be at least about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ point for a particular nucleic acid of the present invention, this occurs, e.g., when a copy of a nucleic acid is created using the maximum codon degeneracy permitted by the genetic code. Stringent hybridization conditions are sequence-dependent and will be different in different circumstances. Longer sequences hybridize specifically at higher temperatures.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook et al., Molecular Cloning: A Laboratory Manual, 3rd Ed., Cold Spring Harbor Laboratory Press, Cold Spring Harbor, N.Y. (2001), for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. In general, a signal to noise ratio of 2× (or higher, e.g., 5×, 10×, 20×, 50×, 100× or more) than that observed for control probe in the particular hybridization assay indicates detection of a specific hybridization. For example, the control probe can be a homologue to a relevant nucleic acid, as noted herein. Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code.

In some embodiments, stringent hybridization conditions include, e.g., 2.0×SSPE (comprising 0.36 M NaCl, 20 mM NaH₂PO₄*H₂O, 2 mM EDTA, pH 7.4) and 0.5% SDS at a temperature of 55° C. and a pH of 7.4. An optimal SSPE range includes, e.g., 1.8 (higher stringency)−2.2×(lower stringency). Varying the percentage of SDS included does not seem to affect stringency. An optimal temperature range includes, e.g., 54-56° C. Assay results typically include light/low signals for high stringency conditions (e.g., 57° C. or above), and additional non-specific signal generally occurs (as well as darker signal) for the low stringency conditions (e.g., 53° C. or below). An optimal pH range includes, e.g., 7.2-7.6. A high stringency condition at, e.g., pH 8.0 or above typically produces a lighter signal, whereas a low stringency condition at, e.g., pH 6.5 or below typically produces a darker signal with an increased level of cross-hybridization.

In contrast, as used herein, the expression “low stringency” denotes hybridization conditions of generally high ionic strength and lower temperature. Under low stringency hybridization conditions, polynucleotides with imperfect complementarity can more readily form hybridization complexes.

Gene: As used herein, the term “gene” most generally refers to a combination of polynucleotide elements, that when operatively linked in either a native or recombinant manner, provide some product or function. The term “gene” is to be interpreted broadly herein, encompassing mRNA, cDNA, cRNA and genomic DNA forms of a gene. In some aspects, genes comprise coding sequences (e.g., an “open reading frame” or “coding region”) necessary for the production of a polypeptide, while in other aspects, genes do not encode a polypeptide. Examples of genes that do not encode polypeptides include ribosomal RNA genes (rRNA) and transfer RNA (tRNA) genes.

The term “gene” can optionally encompass non-coding regulatory sequences that reside at a genetic locus. For example, in addition to a coding region of a nucleic acid, the term “gene” also encompasses the transcribed nucleotide sequences of the full-length mRNA adjacent to the 5′ and 3′ ends of the coding region. These noncoding regions are variable in size, and typically extend on both the 5′ and 3′ ends of the coding region. The sequences that are located 5′ and 3′ of the coding region and are contained on the mRNA are referred to as 5′ and 3′ untranslated sequences (5′ UT and 3′ UT). Both the 5′ and 3′ UT may serve regulatory roles, including translation initiation, post-transcriptional cleavage and polyadenylation. The term “gene” encompasses mRNA, cDNA and genomic forms of a gene.

In some aspects, the genomic form or genomic clones of a gene includes the sequences of the transcribed mRNA, as well as other non-transcribed sequences which lie outside of the transcript. The regulatory regions which lie outside the mRNA transcription unit are sometimes called “5′ or 3′ flanking sequences.” A functional genomic form of a gene typically contains regulatory elements necessary for the regulation of transcription.

“Expression products” are ribonucleic acid (RNA) or polypepetide products transcribed or translated, respectively, from a genome or other genetic element. Commonly, expression products are associated with genes, in some case, genes having biological properties. Thus, the term “gene” can refer to a nucleic acid sequence associated with biological properties, e.g., encoding a gene product with physiologic properties. A gene optionally includes sequence information required for expression of the gene (e.g., promoters, enhancers, etc.).

Gene Expression: The term “gene expression” refers to transcription of a gene into an RNA product (generally in reference to an in vivo process involving an endogenous gene), and optionally incorporate translation into one or more polypeptide sequences. The term “transcription” refers to the process of copying a DNA sequence of a gene into an RNA product, generally conducted by a DNA-directed RNA polymerase using DNA as a template. In vivo, genes frequently display variable expression patterns, that is to say, not all genes are expressed all the time in all cell types, and furthermore, any two genes frequently have different gene expression patterns. For example, a “constitutively-active” or “constitutive-expressed” gene is a gene that is generally expressed in many or most cell types, and generally in a temporally independent manner. In contrast, some genes display “tissue-restricted” or “tissue-specific” expression, where the expression of the gene is limited to a particular cell type or a subset of cell types.

The term “reference sequence” or “reference gene” refers to a nucleic acid sequence serving as a target of amplification in a sample that provides a control for the assay. The reference may be internal (or endogenous) to the sample source, or it may be an externally added (or exogenous) to the sample. Constitutively expressed genes are frequently used as reference genes.

The term “gene expression profile” refers to one or more sets of data that contain information regarding different aspects of gene expression. The data set optionally includes information regarding: the presence of target-transcripts in a cell or cell-derived samples; the relative and absolute abundance levels of target transcripts; the ability of various treatments to induce expression of specific genes; and the ability of various treatments to change expression of specific genes to different levels.

The term “gene expression profile” refers to gene expression data (defined above) collected for a plurality of genes at a give point in time. In some embodiments, “gene expression profile” refers to the particular transcription status, e.g., the “transcriptome,” of a cell or tissue under a given set of physiological conditions. For example, a gene expression profile is characteristic of a particular cell type or particular physiological state. Gene expression profiles can be comparative in nature, for example, comparing the gene expression profiles of a treated versus an untreated cell, or comparing the gene expression profiles of a cancerous cell and a normal or precancerous cell.

Encode: As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In some aspects, the term “encode” describes the process of semi-conservative DNA replication, where one strand of a double-stranded DNA molecule is used as a template to encode a newly synthesized complementary sister strand by a DNA-dependent DNA polymerase.

In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

Isolated: A nucleic acid, protein or other component is “isolated” when it is partially or completely separated from components with which it is normally associated (other proteins, nucleic acids, cells, synthetic reagents, etc.).

Enriched: As used herein, a nucleic acid, protein or other component is “enriched” in a treated heterogeneous mixture when it is relative fraction (i.e., proportion) in the treated heterogeneous mixture is increased compared to its relative fraction in the heterogeneous mixture prior to the treatment (e.g., prior to a purification step). ntext of the invention, one particularly preferred host cell is a pineapple host cell.

Derived from: As used herein, the term “derived from” refers to a component that is isolated from or made using a specified molecule or organism, or information from the specified molecule or organism. For example, a polypeptide that is derived from a second polypeptide can include an amino acid sequence that is identical or substantially similar to the amino acid sequence of the second polypeptide. In the case of polypeptides, the derived species can be obtained by, for example, naturally occurring mutagenesis, artificial directed mutagenesis or artificial random mutagenesis. Mutagenesis of a polypeptide typically entails manipulation of the polynucleotide that encodes the polypeptide.

Similarly, the term “derived from” can apply to polynucleotides. A polynucleotide that is derived from a source polynucleotide can include a nucleotide sequence that is identical or substantially similar to the source nucleotide sequence. In the case of polynucleotides, the derived species can be obtained by, for example, mutagenesis. In some aspects, a derived polynucleotide is generated by placing a source polynucleotide into a heterologous context, i.e., into a context that is different from its native or endogenous context. For example, a gene promoter can be derived from an endogenous gene promoter by removing that endogenous promoter domain and placing it in operable combination with different nucleotide sequences with which it is not normally associated.

Corresponds to: As used herein, the term “corresponds to” or “corresponding to” or similar expressions refer to one component that is related to another component in some significant property. For example, as applied to polynucleotides, a first polynucleotide corresponds to a second polynucleotide if they have identical or nearly identical (or complementary) primary sequence of nucleotide bases. In some aspects, one polynucleotide that is derived from a second polynucleotide corresponds to that second polynucleotide. For example, a PCT amplicon corresponds to the template nucleic acid from which it was amplified. Also, for example, an RNA transcript can correspond to the genomic sequence from which it was transcribed.

Reporter: As used herein, the term “reporter” or equivalent terms refers in a general sense to any component that can be readily detected in a system under study, where the detection of the reporter correlates with the presence or absence of some other molecule or property, or can be used to identify, select and/or screen targets in a system of interest. The choice of the most suitable reporter to use for a particular application depends on the intended use, and other variables known to one familiar with the art. In some aspects, a reporter is a reporter gene.

A wide variety of reporter molecules and genes are known in the art. Each reporter has a particular assay for the detection of that reporter. Some reporter detection assays can be enzymatic assays, while other assays can be immunological in nature, or colorimetric. Further still, a reporter can include, for example, a fluorescent marker (e.g., a green fluorescent protein such as GFP, YFP, EGFP, RFP, etc., or a non-protein fluorescent molecule), a luminescent marker (e.g., a firefly luciferase protein), an affinity based screening marker, or an enzymatic activity.

Expression: The term “expression” refers to the transcription and accumulation of sense mRNA or antisense RNA derived from polynucleotides. Expression may also refer to translation of mRNA into a polypeptide.

Probe: As used herein, the term “probe” refers typically to a polynucleotide that is capable of hybridizing to a target nucleic acid of interest. Typically, but not exclusively, a probe is associated with a suitable label or reporter moiety so that the probe (and therefore its target) can be detected, visualized, measured and/or quantitated. Detection systems for labelled probes include, but are not limited to, the detection of fluorescence, fluorescence quenching (e.g., when using a FRET pair detection system), enzymatic activity, absorbance, molecular mass, radioactivity, luminescence or binding properties that permit specific binding of the reporter (e.g., where the reporter is an antibody). In some embodiments, a probe can be an antibody, rather than a polynucleotide, that has binding specificity for a nucleic acid nucleotide sequence of interest. It is not intended that the present invention be limited to any particular probe label or probe detection system. The source of the polynucleotide used in the probe is not limited, and can be produced synthetically in a non-enzymatic system, or can be a polynucleotide (or a portion of a polynucleotide) that is produced using a biological (e.g., enzymatic) system (e.g., in a bacterial cell).

Typically, a probe is sufficiently complementary to a specific target sequence contained in a nucleic acid to form a stable hybridization complex with the target sequence under a selected hybridization condition, such as, but not limited to, a stringent hybridization condition. A hybridization assay carried out using the probe under sufficiently stringent hybridization conditions permits the selective detection of a specific target sequence.

Label or reporter: As used herein, the terms “label” or “reporter,” in their broadest sense, refer to any moiety or property that is detectable, or allows the detection of, that which is associated with it. For example, a polynucleotide that comprises a label is detectable (and in some aspects is referred to as a probe). Ideally, a labeled polynucleotide permits the detection of a hybridization complex that comprises the polynucleotide. In some aspects, e.g., a label is attached (covalently or non-covalently) to a polynucleotide. In various aspects, a label can, alternatively or in combination: (i) provide a detectable signal; (ii) interact with a second label to modify the detectable signal provided by the second label, e.g., FRET; (iii) stabilize hybridization, e.g., duplex formation; (iv) confer a capture function, e.g., hydrophobic affinity, antibody/antigen, ionic complexation, or (v) change a physical property, such as electrophoretic mobility, hydrophobicity, hydrophilicity, solubility, or chromatographic behavior. Labels vary widely in their structures and their mechanisms of action.

Examples of labels include, but are not limited to, fluorescent labels (including, e.g., quenchers or absorbers), non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like. To further illustrate, fluorescent labels may include dyes that are negatively charged, such as dyes of the fluorescein family, or dyes that are neutral in charge, such as dyes of the rhodamine family, or dyes that are positively charged, such as dyes of the cyanine family. Dyes of the fluorescein family include, e.g., FAM, HEX, TET, JOE, NAN and ZOE. Dyes of the rhodamine family include, e.g., Texas Red, ROX, R110, R6G, and TAMRA. FAM, HEX, TET, JOE, NAN, ZOE, ROX, R110, R6G, and TAMRA are commercially available from, e.g., Perkin-Elmer, Inc. (Wellesley, Mass., USA), and Texas Red is commercially available from, e.g., Molecular Probes, Inc. (Eugene, Oreg.). Dyes of the cyanine family include, e.g., Cy2, Cy3, Cy5, Cy 5.5 and Cy7, and are commercially available from, e.g., Amersham Biosciences Corp. (Piscataway, N.J., USA). For general discussion on the use of flourescence probe systems, see, for example, Principles of Fluorescence Spectroscopy, by Joseph R. Lakowicz, Plenum Publishing Corporation, 2nd edition (Jul. 1, 1999) and Handbook of Fluorescent Probes and Research Chemicals, by Richard P. Haugland, published by Molecular Probes, 6th edition (1996).

Quantitating: The term “quantitating” means to assign a numerical value, e.g., to a hybridization signal fluorescence intensity or a transcript concentration. Typically, quantitating involves measuring the intensity of a signal and assigning a corresponding value on a linear or exponential numerical scale.

Relative Abundance: The term “relative abundance” or “relative gene expression levels” refers to the abundance of a given species relative to that of a second species. The absolute abundance (e.g., the quantitated concentration) does not need to be known. Optionally, the second species is a reference sequence.

Correlate: As used herein, the term “correlate” refers to making a relationship between two or more variables, values or entities. If two variables correlate, the identification of one of those variables can be used to determine the value of the remaining variable.

Sample: As used herein, the term “sample” is used in its broadest sense, and refers to any material subject to analysis. The term “sample” refers typically to any type of material of biological origin, for example, any type of material obtained from animals or plants. A sample can be, for example, any fluid or tissue such as blood or serum, and furthermore, can be human blood or human serum. A sample can be cultured cells or tissues, cultures of microorganisms (prokaryotic or eukaryotic), or any fraction or products produced from or derived from biological materials (living or once living). Optionally, a sample can be purified, partially purified, unpurified, enriched or amplified. Where a sample is purified or enriched, the sample can comprise principally one component, e.g., nucleic acid. More specifically, for example, a purified or amplified sample can comprise total cellular RNA, total cellular mRNA, cDNA, cRNA, or an amplified product derived there from. In some aspects, the sample can be a degraded RNA sample, e.g., an RNA sample derived from formalin-fixed, paraffin-embedded tissues.

The sample used in the methods of the invention can be from any source, and is not limited. Such sample can be an amount of tissue or fluid isolated from an individual or individuals, including, but not limited to, for example, skin, plasma, serum, whole blood, blood products, spinal fluid, saliva, peritoneal fluid, lymphatic fluid, aqueous or vitreous humor, synovial fluid, urine, tears, blood cells, blood products, semen, seminal fluid, vaginal fluids, pulmonary effusion, serosal fluid, organs, bronchio-alveolar lavage, tumors, paraffin embedded tissues, etc. Samples also can include constituents and components of in vitro cell cultures, including, but not limited to, conditioned medium resulting from the growth of cells in the cell culture medium, recombinant cells, cell components, etc.

Kit: As used herein, the term “kit” is used in reference to a combination of articles that facilitate a process, method, assay, analysis or manipulation of a sample. Kits can contain written instructions describing how to use the kit (e.g., instructions describing the methods of the present invention), chemical reagents or enzymes required for the method, primers and probes, as well as any other components. In some embodiments, the present invention provides kits for amplifying members of a population of degraded nucleic acids in a sample, for determining nucleic acid quality in a nucleic acid sample, for producing a gene expression profile from a degraded RNA sample, measuring gene expression values within degraded RNA samples, and measuring DNA or RNA degradation as a function of relative amplification efficiency. These kits can include, for example but not limited to, reagents for sample collection, reagents for the collection and purification of RNA, a reverse transcriptase, primers suitable for reverse transcription and first strand and second strand synthesis to produce a target amplicon, a thermostable DNA-dependent DNA polymerase and free deoxyribonucleotide triphosphates. In some embodiments, the enzyme comprising reverse transcriptase activity and thermostable DNA-dependent DNA polymerase activity are the same enzyme, e.g., Thermus sp. ZO5 polymerase or Thermus thermophilus polymerase.

Solid support: As used herein, the term “solid support” refers to a matrix of material in a substantially fixed arrangement that can be functionalized to allow synthesis, attachment or immobilization of polynucleotides, either directly or indirectly. The term “solid support” also encompasses terms such as “resin” or “solid phase.” A solid support may be composed of polymers, e.g., organic polymers such as polystyrene, polyethylene, polypropylene, polyfluoroethylene, polyethyleneoxy, and polyacrylamide, as well as co-polymers and grafts thereof. A solid support may also be inorganic, such as glass, silica, silicon, controlled-pore-glass (CPG), reverse-phase silica, or any suitable metal. In addition to those described herein, it is also intended that the term “solid support” include any solid support that has received any type of coating or any other type of secondary treatment, e.g., Langmuir-Blodgett films, self-assembled monolayers (SAM), sol-gel, or the like.

Array: As used herein, “array” or “microarray” is an arrangement of elements (e.g., polynucleotides), e.g., present on a solid support and/or in an arrangement of vessels. While arrays are most often thought of as physical elements with a specified spatial-physical relationship, the present invention can also make use of “logical” arrays, which do not have a straightforward spatial organization. For example, a computer system can be used to track the location of one or several components of interest that are located in or on physically disparate components. The computer system creates a logical array by providing a “look-up” table of the physical location of array members. Thus, even components in motion can be part of a logical array, as long as the members of the array can be specified and located. This is relevant, e.g., where the array of the invention is present in a flowing microscale system, or when it is present in one or more microtiter trays.

Certain array formats are sometimes referred to as a “chip” or “biochip.” An array can comprise a low-density number of addressable locations, e.g., 2 to about 10, medium-density, e.g., about a hundred or more locations, or a high-density number, e.g., a thousand or more. Typically, the chip array format is a geometrically-regular shape that allows for facilitated fabrication, handling, placement, stacking, reagent introduction, detection, and storage. It can, however, be irregular. In one typical format, an array is configured in a row and column format, with regular spacing between each location of member sets on the array. Alternatively, the locations can be bundled, mixed, or homogeneously blended for equalized treatment or sampling. An array can comprise a plurality of addressable locations configured so that each location is spatially addressable for high-throughput handling, robotic delivery, masking, or sampling of reagents. An array can also be configured to facilitate detection or quantitation by any particular means, including but not limited to, scanning by laser illumination, confocal or deflective light gathering, CCD detection, and chemical luminescence. “Array” formats, as recited herein, include but are not limited to, arrays (i.e., an array of a multiplicity of chips), microchips, microarrays, a microarray assembled on a single chip, arrays of biomolecules attached to microwell plates, or any other appropriate format for use with a system of interest.

High Throughput: The term “high throughput format” refers generally to a relatively rapid completion of an analysis.

Highly Parallel: The term “highly parallel” refers to the simultaneous processing and/or analysis of many samples.

Platform: The term “platform” refers to the instrumentation method used for sample preparation, amplification, product separation, product detection, or analysis of data obtained from samples. The term “miniaturized format” refers to procedures or methods conducted at submicroliter volumes, including on both microfluidic and nanofluidic platforms.

### BRIEF DESCRIPTION OF THE FIGURES

FIGS. 1A, 1B and 1C provide three Agilent Technologies Bioanalyzer analyses of three progressively degraded total RNA samples. Data is presented pictorially as electropherogram. These systems provide quantitative data on the relative amounts of RNA present at a range of molecular sizes.

FIG. 2 provides an illustration of chimeric primers utilizing gene-specific sequence and universal priming sequence in an RT-PCR reaction strategy.

FIG. 3 provides an illustration of a multiplex RT-PCR reaction.

FIG. 4 provides a bar graph illustrating the dynamic range of RNA detection (by fluorescence) using KanR transcripts (×1000).

FIG. 5 provides a bar graph illustrating relative gene expression responses of 21 different transcripts in primary rat hepatocytes to three different glitazone treatments.

FIG. 6 provides a bar graph illustrating the relative heme oxygenase (HO1) gene expression response in primary rat hepatocytes to three different glitazone treatments.

FIG. 7 provides a bar graph illustrating relative gene expression responses of 21 different transcripts in clone9 rat hepatocyte cells to three different glitazone treatments.

FIG. 8 provides cluster data for SRBCTs in 20 different tumor samples for 33 different gene transcripts.

FIG. 9 provides an illustration of the proximal-primer, multiplexed PCR (PPM-PCR) method strategy, showing that various lengths of gene-specific sequence and supplemental spacer sequences can be used to generate short amplicons of differing lengths.

FIG. 10 provides a capillary electrophoresis trace from a multiplex RT-PCR raction containing 10 different target amplicons illustrated in FIG. 9.

FIG. 11 provides the pool ratios of experimental RNA samples.

FIG. 12 provides a schematic overview of the Affymetrix protocol for expression analysis using microarrays.

FIGS. 13A through 13D provide the results of an analysis of the performance of PPM-PCR and UPM-PCR multiplexed PCR formats with fresh tissue versus FFPE-derived RNA.

FIG. 14 provides an illustration of RNA metric analysis illustrating the impact of degraded RNA on PCR signal as a function of amplicon size.

FIG. 15 provides a table showing data from a microarray and UMP-PCR concordance analysis, where male rats were treated with doses of clofibrate, and relative changes in expression of various genes were observed using the two different methodologies. Table adapted from Auer and Lyianarachchi, 2003 “Chipping away at the chip bias: RNA degradation in microarray analysis,” Nature Genetics 35(4):292-293.

FIG. 16 provides a table showing the results of expression analysis of tissue-specific genes.

FIG. 17 provides the results of a PPM-PCR analysis of breast tissue for 18 different transcripts in normal and cancer tissue samples. Results are shown as normalized expression relative to a GAPD reference gene.

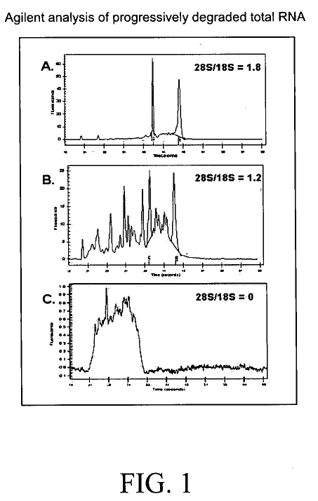

### DETAILED DESCRIPTION OF THE INVENTION

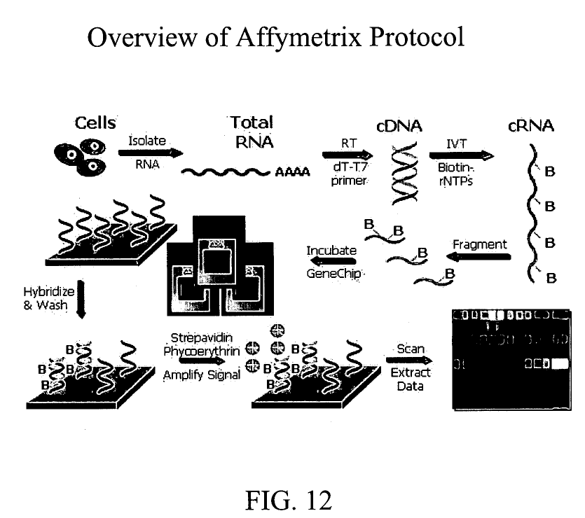

The present invention provides new PCR-based methods for amplifying degraded nucleic acids, accurately and directly measuring the level of degradation within a nucleic acid population (e.g., mRNA or total cellular RNA), provides a gene-level metric of nucleic acid (e.g., RNA) quality, and provides methods for producing gene expression profiles using degraded RNA starting material. The invention can also be applied to the analysis of DNA samples. This invention provides new methods which are improvements over the art and achieves the goals of (i) providing a system for assessing the quality of nucleic acid (e.g., DNA or RNA) that can be derived from, for example, formalin-fixed, parafin-embedded (FFPE) tissue samples, and furthermore, using this assessment to ascertain the suitability of a nucleic acid sample for use in a microarray experimental system, e.g., using Affymetrix® GeneChip® microarrays (see FIG. 12); and (ii) using proximal-primer, multiplexed-PCR (PPM-PCR) based tools to measure, confirm and/or validate selected gene measurements.

In some aspects, the invention provides researchers with a set of tools that will allow them to mine the vast collections of existing FFPE samples for the discovery of new gene expression biomarkers for improved diagnosis and prognosis of cancer and other diseases. This approach can be used to yield valuable information, for example, in the analysis of cancer genetics, e.g., comparing data from fresh frozen and previously stored fixed prostate cancer-associated tissue samples. The stored tissue samples can be any age, for example, ranging from 6 months to 15 years in age. Such a study can include analysis of key prostate specific cancer genes.

The present invention provides improved and innovative approaches to assessing the quality of RNA for use in both microarray and PCR-based studies. In some embodiments, the approach focuses on PCR methods for assessing the integrity of a sampling of RNA transcripts. The methods described herein provide improvements over known methods in the art in several aspects, including (a) the utilization of highly multiplexed PCR amplification to increase the number of genes to be sampled while decreasing the number of reactions, (b) utilizing multiple primer sets to generate size ranged (e.g., small, mid and large) amplicons for each gene to provide transcript length integrity information to the data set, (c) analysis of constitutively expressed genes providing broad application over multiple tissue types, and (d) selection of targeted amplicon sequences based on relative proximity to the probe sequences in a gene set microarray, e.g., the Affymetrix® GeneChip® probe set as shown in FIG. 12 for each gene. In some aspects, the methods of the invention have the ability to evaluate and determine RNA sample quality by directly examining a subset of transcripts; the methods of the invention have the ability to identify useful RNA samples, e.g., RNA samples derived from FFPE tissues that can be used in global expression analysis.

Universal-Primer-Based, Multiplexed RT-PCR (UPM-PCR)

Critical to the performance of this invention is the development of novel, highly multiplexed methods of PCR amplification. These methods build on the fundamental strengths of the polymerase chain reaction to selectively amplify gene targets in a highly predictable and reproducible fashion, and use a novel, universal-primer-coupled RT-PCR (UPM-PCR) strategy to create quantitative methods for gene expression analysis. Moreover, in some aspects, these methods accommodate large numbers of primer pairs in a single multiplexed reaction. However, it is not intended that the number of transcripts targeted in a UPM-PCR analysis be particularly limited. For example, between 2 and about 100 primer pairs can be used to target the various transcripts in a sample. Alternatively, between about 10 and about 100 primer pairs can be used to target the various transcripts. In some embodiments, approximately 35 primer pairs are used to target the various transcripts.

In some embodiments, the UPM-PCR method utilizes a universal primer scheme to lock in the relative ratios of the genes in the multiplex reaction as they are amplified. Each of the different primer sets (for example, each of 35 different primer sets) determines a different sized amplification product. The set of amplicons can be resolved and quantified by any suitable method, for example, but fluorescence capillary electrophoresis. Advantages of the method, and in particular the fluorescence capillary electrophoresis methologies, include (a) high levels of multiplexing, for example, at least 2, at least 10, at least 35, at least 40, or at least 100, genes targeted per PCR reaction; (b) 3 plus logs of working dynamic range; (c) good reproducibility with mean Coefficients of Variance (CV) under 10%; (d) high sensitivity, capable of detecting single copy per cell transcripts using as little as 5 ng of total RNA per reaction; (e) low cost per assay via the use of standard, off-the-shelf reagents and equipment; (f) compatibility with assays performed in 96 and 384-well format and throughputs of hundreds of samples per day; and (g) a fast assay development cycle. The generic nature of the assay platform means that a new assay for a given set of genes can be quickly developed within a very short time.

The UPM-PCR methods of the invention are not limited to capillary electrophoresis as an analysis endpoint. As with other PCR and non-PCR amplification strategies, there are a range of other analytical techniques as known to one skilled in the art for detecting and quantitating nucliec acid (e.g., DNA) fragments, including multiple types of hybridization and capture systems such as bead-based monitoring via flow cytometry or confocal scanning, one, two and three dimensional nucleic acid microarray systems, other chromatographic methods for separating and detecting PCR amplicons such as HPLC, micro and nanofluidic nucleic acid separation devices, and mass spectrometry. Alternatively, multiplex reactions can be divided and a plurality of probe-based methods can be used for detecting specific nucleic acids both in solution or on a solid phase.

Standard multiplex RT-PCR is not typically quantitative, especially with very low concentrations of RNA. Significant biases can be introduced during the exponential amplification that lead to varied and nonreproducible data. These biases result from primer-primer interactions, primer-product cross-reactions, and from concentration and sequence-dependent variations in amplification efficiency, most notably seen in the latter part or plateau phase of thermal cycling. In some (but not all) embodiments, the UPM-PCR processes of the invention convert multiplexed PCR reactions to a two-primer process using universal priming strategy with universal primers to overcome these deficiencies.

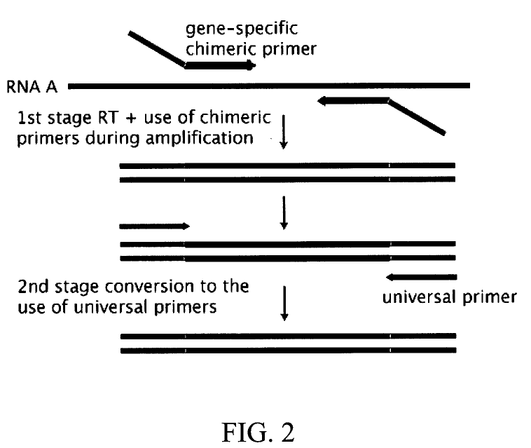

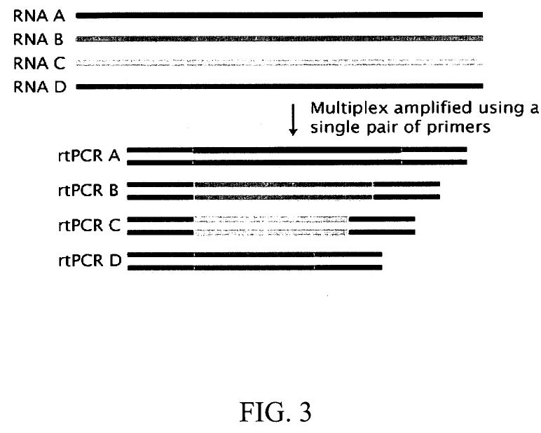

Key to the conversion process to a universal primed multiplex system, is the use of chimeric gene-specific primers, as outlined in FIGS. 2 and 3. The reaction starts by using gene-specific primer sets that are capable of specifically detecting each target mRNA. As shown in FIG. 2, these gene-specific primers carry on their 3′ ends the gene-specific target sequences, and carry on their 5′ ends a consensus or universal primer sequence. The same universal priming sequence can exist on both the forward and reverse primers, or alternatively, different left and right universal priming sequences can be used. During the first few cycles of amplification the specific mRNA targets are copied by these chimeric primers, creating double-stranded cDNA products that are extended (tailed) with the universal priming sequence. The reactions all contian the pair of universal primers present at significantly higher concentrations, e.g., a chimeric gene-specific primer to universal primer ratio (chimeric-gene-specific:universal ratio) of between about 1:2 and about 1:100, or alternatively between about 1:10 and about 1:100; or alternatively, at about 1:50 (0.02 μM gene specific versus 1 μM universal). Therefore, as PCR progresses, the amplification is quickly taken over by the single pair of universal primers. In fact, the chimeric, gene-specific primers are only significantly involved during the reverse transcription and second strand cDNA synthesis steps. This transition from the use of many primers to only two effectively collapses the level of reaction complexity and locks in the relative concentrations of the different gene targets. In the universal primer amplification reaction all the products are effectively the same chemical species and are not differentially amplified, and the relative gene ratios can be maintained even as the reaction reaches the near plateau phase. Unlike real-time PCR methods like TaqMan®, UPM-PCR does not necessarily require use of a probe. As a consequence, the PCR primers can be placed with their 3′ ends as close as a few bases from each other, leading to amplicons using as little as 40 bases of message sequence. In contrast, TaqMan® requires at least 70 bases and even this short length is very challenging from a design perspective. The present invention explicitly takes advantage of this capability to use small amplicons to amplify the degraded RNA.

The reaction depicted in FIG. 2 can be simultaneously employed for a multiplicity of RNA targets, i.e., a multiplex reverse transcriptase PCR reaction is established, as shown in FIG. 3. The initially generated gene-specific sequences are amplified by use of the universal primers. FIG. 3 shows four transcripts (A through D) for illustration purposes, but the actual number can be much larger, for example, at least 10, at least 20, at least 30, at least 40, at least 50, at least 60, at least 70, at least 80, at least 90, or at least 100 or more RNA targets can be simultaneously amplified in the multiplex PCR reaction.

In some embodiments, capillary electrophoresis is used to detect the multiple amplicons following the UMP-PCR reaction. In this case, amplicons can be engineered to be distinguishable from each other by their length. This can be accomplished in various ways.

In some embodiments, the target amplicon length is determined by the amount of target sequence (e.g., the degraded nucleic acid) that is amplified by the chimeric gene-specific primers. In some embodiments, it is preferable for a subset of the amplicons or essentially all of the amplicons to be small in size. For example, an amplicon can comprise not more than about 200 base pairs of nucleotide sequence corresponding to the target molecule. In still other embodiments, an amplicon can comprise not more than about 100 base pairs, or not more than about 80 base pairs, or about not more than about 60 base pairs of nucleotide sequence corresponding to the target molecule. These sizes above to do not incorporate the nucleotides that are added to the amplicon from the universal priming sequences.

In other embodiments, the target amplicon length is controlled by the addition of at least one “spacer” nucleotide between the target-specific portion of the chimeric primer and the universal sequence in the chimeric primer. There is no limitation on the number of nucleotides that might be added to an amplicon in this manner, with the end result being that the multiple amplicons generated in the multiplex PCR reaction (UMP-PCR) have different lengths that can be differentiated from each other (e.g., by their resolution in capillary electrophoresis). The addition of these spacer nucleotides is a useful tool in the fine-tuning required to achieve amplicons with resolvable size differences.

With the concentrations of the gene-specific primers kept low their participation in cross-reactions and mis-reactions is limited. This leads to a higher probability of success in amplification and a significantly reduced likelihood for creating artifacts. First pass success rates for primer design can be greater than 90%.

Using a single label on the forward universal primer, the PCR products can be analyzed using a fluorescence capillary electrophoresis system, e.g., the ABI PRISM® 3100 Genetic Analyzer or the Beckman-Coulter™ CEQ™ 8800 Genetic Analysis System. Post amplification, the different gene amplicon products can be differentiated and quantified by electrophoresis because each pair of gene-specific primers has been designed to generate a different size PCR product.

One of skill in the art is well familiar with regents, instrumentation, and the variables that may be adjusted to optimize a multiplex PCR reaction. Descriptions of multiplex reaction conditions and reagents (including gene targets, gene specific primers, and universal priming sequences) are well known to one of skill. Indeed, the art has reported more than 1,000 target genes and more than 100 different multiplex reactions. See, for example, but not limited to, Ferre et al., (1996) Quantitation of RNA Transcripts Using RT-PCR: A Laboratory Guide to RNA: Isolation, Analysis, and Synthesis (ed. Krieg) 175-190 (Wiley-Liss Publishers, NY); Kramer et al., (2003) “Transcription profiling distinguishes dose-dependent effects in the livers of rats treated with clofibrate,” Toxicol Pathol 31:417-431; and Johnson et al., (2002) “Multiplex gene expression analysis for high-throughput drug discovery: screening and analysis of compounds affecting genes overexpressed in cancer cells,” Mol Cancer Ther 1:1293-1304. It is not intended that the multiplex reactions of the present invention be limited to any type of instrumentation or reagents, particular gene target sequences, any particular primer sequences, or any particular methods of amplicon identification or detection or identification. Alternative methodologies and reagents known in the art in addition to those disclosed herein are intended to be within the scope of the invention.

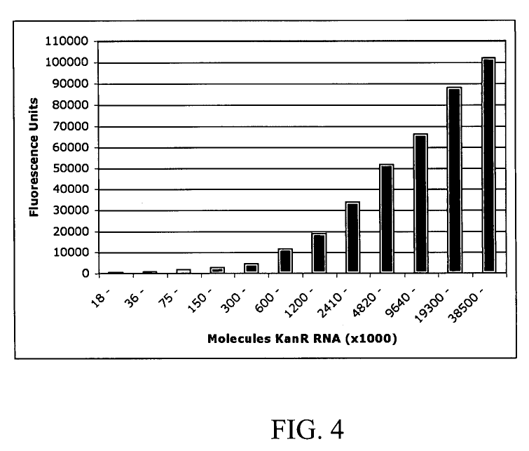

The UPM-PCR approach shows a wide, workable dynamic range for measuring expression change. To assess the dynamic range of RNA detection by the assay, purified kanamycin resistance mRNA, also used as an external control, was spiked into 20 ng of total RNA from cultured HepG2 cells in the range of 18,000 to 38 million molecules (0.03 to 64 attomoles). The results show a smooth response over a greater than 3 log range (see FIG. 4). This range permits the simultaneous measurement of both high and low copy number transcripts in a single multiplex PCR reaction. This experiment also demonstrates that the minimum detectable level of spiked KanR mRNA that could be distinguished from zero was 30 zeptomoles, or 18,000 molecules. Thus the assay can detect on the order of one transcript copy per cell using 10⁴ cells, which is less than 1% of the cells typically present in a prostate needle biopsy, for example. This sensitivity level makes it possible to run many multiplex reactions using only a very small amount of total RNA, and enables us to measure expression values for hundreds of genes. Thus, the UPM-PCR protocol can effectively utilize a wide variation in RNA concentration (e.g., 5-50 ng) and still provide comparable data.

It is not intended that any method of the invention be limited to any particular software or hardware to practice the invention. Various software tools can assist in execution of the methods of the invention. For example, useful software tools include (but not limited to) software for design of assays and the management of data flow related to running gene expression studies in 96-well and 384-well formats. For example, software tools for any method of the invention can be employed for automated: (a) primer design/selection and multiplex assembly, most importantly so that all of the products are of different length and resolvable by capillary electrophoresis, (b) project and reaction plate setup (c) data collection and sample mapping, (d) data checking, and (e) first pass data analysis. Some software used with the invention, e.g., JAVA-coded tools, communicate and store data via an Oracle® database and middleware. The software can be platform independent with implementations running in Linux, Microsoft and/or Apple system environments. One of skill is familiar with a wide array of software products that can be used in conjunction with the invention.

UMP-PCR and Microarray Concordance

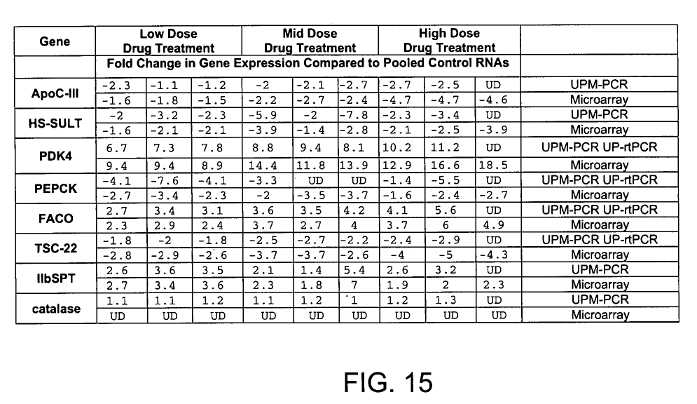

Comparisons have been performed between UPM-PCR and microarrays demonstrating good correlation between the two methods. See, e.g., Auer and Lyianarachchi, 2003 “Chipping away at the chip bias: RNA degradation in microarray analysis,” Nature Genetics 35(4):292-293. In this experiment, male rats were treated in triplicate with clofibrate at 200 (low dose), 400 (mid dose) and 800 (high dose) mg/kg/day for five days. Clofibrate acts as an agonist for the peroxisome proliferator activated receptor (PPAR-α). Total RNA was isolated and prepared either for hybridization to a rat cDNA microarray or for UPM-PCR. FIG. 15 shows a table of data adapted from Auer and Lyianarachchi for a selected set of genes that were run with both assays. In the UPM-PCR these genes were run as part of a 21 gene multiplex reaction (i.e., a 21-plex). Values shown are fold change versus pooled control RNAs. There is good correlation both in terms of direction of change and strength. The calculated R² value was 0.89 with the slope of 1.1, indicating that there was 89% concordance between the two platforms. Also of note is the catalase gene (CAT), which was undetectable in the microarray but could be detected and shown to be virtually unchanged by the UPM-PCR method.

EXAMPLES 1 and 2 provide two demonstrations of the UMP-PCR method.

Proximal-Primer Multiplexed PCR (PPM-PCR)

In the development of methods to fully analyze RNA gene expression levels from degraded RNA, it is necessary manipulate the multiplexed PCR method down toward the smallest PCR amplicons that can be generated and resolved. In a standard two-primer PCR reaction the practical limit is amplicons down to about 40 base pairs in length. This is well below what can be achieved using real-time PCR methods such as TaqMan®. Because TaqMan® requires that the amplicon include room for the labeled probe, it is necessary to add at least another 30 base pairs of sequence to the size of the amplicon, generating a minimum 70 base pair amplicon. This 70 base pair limit is very difficult to achieve in real-time detection methods, where the minimum amplicon size is typically 80-120 base pairs, depending on the gene.

The UPM-PCR methods described herein offer advantages over TaqMan® in that it does not require the use of a probe and as such is able to work in the 40-60 base pair amplicon range quite readily. Note that the final amplicon size has an additional 40 base pairs of universal sequence, but this sequence does not impact the ability to amplify from very small runs of mRNA sequence. In some embodiments, the UPM-PCR method requires that the amplicon for each gene differ in size so that all of the products can be separated and detected via capillary electrophoresis. To accommodate this need for size differentiation and to enable the amplicons to stay in the 40-60 base pair range, the invention provides a novel variant of the UPM-PCR method, described as Proximal-Primer, Multiplexed PCR (PPM-PCR).

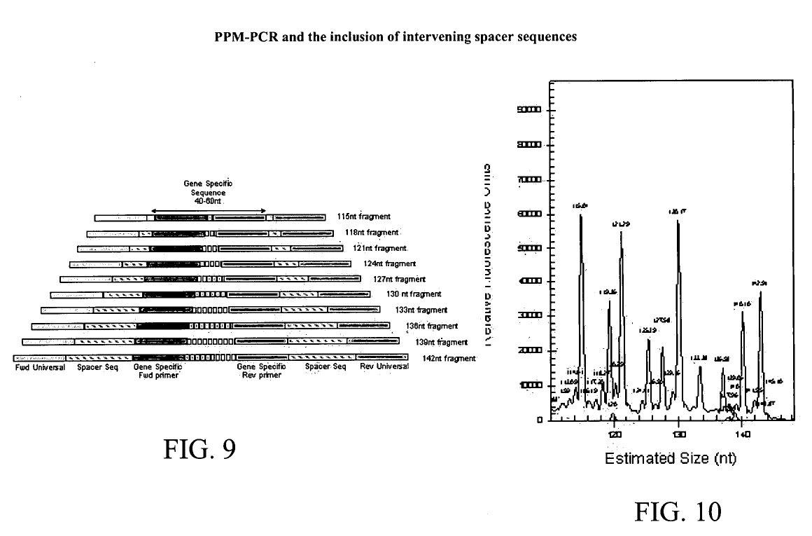

It is the objective of the PPM-PCR methods that a multiplexed PCR is performed in such a manner that the 3′-ends of the forward and reverse primers in each primer pair is in close proximity of each other, e.g., 20 bases or less from each other. To achieve this, additional sequence can be optionally appended to the forward and reverse primers in the form of the universal primer sequences (and optionally spacer nucleotides). The use of chimeric primers provides the opportunity to optionally add in additional intervening spacer sequence between the gene specific primer and universal primer regions of the chimeric primers. FIG. 9 schematically outlines PPM-PCR strategy where one can see that it is relatively straightforward to assemble a 10-plex with sizes ranging from 115 to 142 total base pairs in size. Shown in FIG. 10 is a capillary electrophoresis trace from a multiplex containing 10 different reference genes, as listed in TABLE 1.

TABLE 1
PPM-PCR Reference Genes

| Gene | GenBank Accesion No. |
|---|---|
| GAPD | NM_002046 |
| HPRT1 | M31642 |
| CycloA | BC000689 |
| GK | X68285 |
| TRFR | BC001188 |
| B2M | BC032589 |
| ARP | NM_203388 |
| TAF7 | X97999 |
| GUSB | NM_000181 |
| bAct | NM_001101 |

Each of these target primer pairs amplifies 60 bases or less of target mRNA sequence. The PCR products are all designed to have a four base pair separation from the adjacent products and all can be easily resolved to base line. In initial studies, low background and minimal amplification artifacts were observed down to approximately 80 base pairs. The reduced distance between multiplex fragments and the apparent ability to detect fragments down to 80 base pairs suggest that this format can accommodate 20 or more multiplex genes. To further extend the multiplexing, two different dyes are utilized during amplification with half of the products being labeled with a first dye and the other half being labeled by a second dye. The two dye system uses a three universal primer strategy wherein two different forward universal primers are used carrying the two different dyes. For example, this strategy enables the development multiplexes up to or exceeding 40 genes, with all genes being amplified using 60 bases or less of target sequence.

Although a one or two dye system is described herein, it is not intended that the invention be limited to one or two dye detection systems. Indeed, any multiplicity of dyes can be used in the multiplex reactions. Indeed, as the number of target genes that are simultaneously analyzed in the multiplex reactions increases, it is advantageous to incorporate pluralities of dyes for the labeling of the amplicons to allow discrimination of a larger number of amplicons, e.g., by capillary electrophoresis. One of skill in the art knows well the wide array of dyes (fluorescent dyes) and other types of labels that can be used in such methods.

Development of Multiplexed RT-PCR RNA QC Assays

The invention provides methods for the assessment of RNA quality (an “RNA QC assay”) in a sample, e.g., in mRNA samples of expressed genes. These methods incorporate multiplexed PCR assays that generate a plurality of resolvable (e.g., different sized) amplicons from the same target gene, and furthermore, optionally do so from a plurality of target genes. The amplicons generated from such reactions can be used as a quantitative or qualitative metric for nucleic acid (e.g., RNA) integrity.

Each of the RNA target genes in the RNA QC assay has at least two separate regions chosen and amplified with at least two primer pairs that generate either a short product (for example, as short as about 40 base pairs of target sequence not counting base pairs from spacer, universal primer or other non-target sequence) or a relatively longer product (for example, a product as long as 200 base pairs of target sequence not counting base pairs from spacer, universal primer or other non-target sequence). In some embodiments, a third primer pair that generates an intermediate length third product can also be used. In some embodiments where three primer pairs are used to generate three amplicons from the same transcript, the three amplicons can have a size ranges of, for example, between about 40-60 base pairs, between about 100-120 base pairs, and between about 180 and 200 base pairs. Amplicons from the same transcript can contain overlapping sequence, or alternatively, can be derived from non-overlapping regions in the target transcript of interest. In some embodiments, the 3′-end of one primer (e.g., the forward primer) is not more than 20 base pairs from the 3′-end of the other primer (e.g., the reverse primer). An amplicon thus produced typically comprises not more than 60 base pairs of target nucleotide sequence, where 20 base pairs of target sequence comes from each of the PCR primers, and an additional not more than 20 base pairs originates from the nucleotide sequence that lies between the 3′-ends of the two primers when the primers are hybridized to DNA.

In these RNA QC methods of the invention, the primer sets can be combined in a single multiplex reaction, or alternatively, can be used in multiple reactions. In some embodiments, the multiplexing reactions in the RNA QC methods can utilize the universal primed, multiplexed RT-PCR method (UPM-PCR) that can quantitatively analyze a plurality of genes (e.g., 20-30 genes) per reaction with minimal amplification artifacts.

It is not intended that the number of gene targets that are amplified in a RNA QC assay multiplex reaction is especially limited. In some aspects, the number of targets amplified is limited only by the number of amplicons that can be resolved by whatever readout is used (e.g. capillary electrophoresis). In some embodiments, only two amplicons are generated from only one target transcript in the multiplex amplification reaction. In some embodiments, more than two target transcripts is used, e.g., 5 or more, 10 or more, 15 or more, 20 or more, 25 or more, 30 or more, 35 or more, 40 or more, 45 or more, 50 or more, 75 or more, or 100 or more. In some embodiments, between about 2 and 100 target transcripts are used in the multiplex reaction, or between about 10 and 40 targets, or between 20 and 30 targets, or between 30 and 40 targets, are used.

Furthermore, it is not intended that the particular gene targets for amplification in the multiplex reactions be limited in any way. For example, the multiplex reactions can target any transcripts of interest. In the methods of the invention, target transcripts can include reference invariant pathway associated, cell type associated, tissue type associated, disease associated, and drug responsive genes. The reference genes or invariant genes can be, for example, constitutively expressed, housekeeping and/or tissue-specific genes that are expressed at low, mid and/or high levels.

An aspect in the development of the multiplex PCR RNA QC assay is the selection of the gene targets. Generally, genes that are expressed at a range of levels within the target sample as used. In some embodiments of the RNA QC assays of the invention, target genes can be selected from constitutively expressed “reference” genes and/or tissue specific genes.

In addition to being well represented in multiple tissue types, reference genes tend to maintain very consistent levels of expression from sample to sample and individual to individual. This constitutive and stable expression provides a baseline, in terms of relative gene ratios, that can be used as part of the algorithm to score the integrity of the RNA and help understand the relative impacts of gene-specific versus global mechanisms of RNA degradation. A second type of gene that can be present are cell, tissue or organism specific transcripts. The use of reference genes provides the benefit of wide applicability in a broad range of applications and tissue types. A wide variety of reference genes are known in the art and which find use with the methods of the invention. A representative list of reference genes is provided in Table 2.

TABLE 2
Reference Genes
β-2-microglobulin
Hypoxanthine ribosyl transferase
Transferrin Receptor
Transcription Factor IID
18S rRNA
Acidic Ribosomal Protein
glycerol kinase
β-glucuronidase
Lysosomal hyaluronidase
Proteasome subunit Y
Elongation factor EF-1-alpha
Ribosomal protein L37a (RPL37A)
Ca₂-activated neutral protease large subunit
18kDa Alu RNA binding protein
Nuclear factor NF45
E2 Ubiquitin conjugating enzyme UbcH5B
Histone deacetylase HD1
Ezrin
QRSHs glutaminyl-tRNA synthetase
16S rRNA
MLN51
ATP synthase
cyclophilin A
β-actin
GALPDH

The reference genes used in the methods of the invention (i.e., the genes shown in Table 2) can be genes classically used as reference genes, e.g., β-actin and GAPDH, or have been identified through tissue surveys using global RNA surveys such as those using Affymetrix® microarrays (see, e.g., Warrington et al. (2000) “Comparison of Human Adult and Fetal Expression and Identification of 535 Housekeeping/Maintenance Genes,” Physiol. Genomics 2:143-147).

It is not intended that the reference genes used in the methods of the invention be limited to the reference genes shown in Table 2, as one of skill in the art recognizes that other reference genes can also be used. Furthermore, it is not intended that the genes used in the RNA QC assays of the invention be limited to constitutively expressed reference genes, as other gene types can also be used.

Assay Design

In some embodiments, the RNA QC assays of the invention focus on a specific size range of amplicon to generate, e.g., short or long, and a selected region of the transcript to amplify, e.g., 5′, middle, and/or 3′regions. The amplicons generated make use of the UPM-PCR and PPM-PCR techniques to assure that all of the amplicons require, e.g., about 40-60 bases of target sequence, while the long amplicons require longer target sequences, e.g., about 180-200 bases of target sequence or any linger length that can be resolved from the shorter amplicon. Amplicons of intermediate length can also be generated, e.g., requiring about 100-120 base pairs of target. The experimental Type 1 and Type 2 RNAs described in EXAMPLE 3 can be used to test and verify assay design

In selecting the target regions to amplify, various factors can be taken into consideration, e.g., message length and locations of commercial probes (e.g., Affymetrix® probes) for each gene. Depending on the gene, the full length transcript can vary by many thousands of bases in size. However, in some Affymetrix® microarray systems, for example, all of the probes are biased toward the 3′end. This is not surprising given that one or more common amplification and labeling technique is initiated by making cDNA using a polyT primer approach. To balance out these two somewhat conflicting factors, a “middle region” can be selected for amplification, where the “middle region” to be amplified is a position relative to the Affymetrix® probe set for that gene (not the middle of the transcript). Essentially, the 3′ and middle amplicons can be used to flank the Affymetrix® sequences, being placed, for example within 100-200 bases upstream and downstream from the probe set, respectively. The 5′-amplicons can be targeted to center around, e.g., 200-300 bases from the 5′-end.

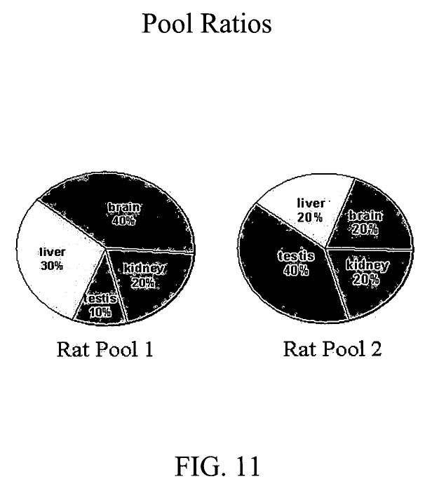

Following the in silico design phase, all of the gene specific primer sequences can optionally be synthesized with a universal primer sequence tail on the 5′-end. The testing process can optionally involve a first step of testing primer pairs individually with RNA pools (see, e.g., Example 3 and FIG. 11) and a panel of tissue specific RNA isolates, e.g., a number of different human RNAs including prostate and the different tissue titration RNAs. Following the individual primer pair testing a second step of testing the fully assembled multiplex can optionally be undertaken. Typical failures might be seen in the first step with no PCR product detected. Generally, within the first pass greater than 90% of the genes that are initially designed are detectable as singlets and within the multiplex. A second pass effort can be made to include the genes that did not make the first pass. This process can optionally involve a second round of primer design and selection of primers that generate appropriately sized products, i.e. they do not overlap in size with existing PCR products that comprise the multiplex. A small percent of the time, one or more genes cannot be made to work within the multiplex. This is not unusual for PCR-based methods and is usually the result of poor sequence information, unusual sequence structure or the lack of expression of the mRNA within the test samples. Fortunately, this type of failure is unlikely in the methods of the invention, since the sequences of the reference genes typically finding use in the methods are well documented.

Final qualification of the assays can be optionally performed by analyzing the full series of tissue titration RNA samples, e.g., titrations of RNAs from two tissue sources. As previously described, each of the sample pairs expresses each of the genes at different levels so that a titration of the two RNAs can reveal a linear progression in the expression level of each gene from 100% sample A to 100% sample B. For invariant reference genes it is expected that these levels change from very low or no expression to relatively high expression. For the other reference genes the progressive change is more subtle but still detectable for most in the appropriate tissue pair. This kind of testing may occasionally reveal a problem gene, e.g., <1% of the time, and this gene can either be replaced with a different gene, removed from the multiplex or undergo redesign.

Generally, by observing the relative efficiencies in the generation of the short and long amplicons from the same gene (and optionally one or more intermediate sized amplicons from the same gene), the degree of degradation can be qualitatively observed, where genes showing little or no difference in the molar concentrations of the short and long amplicons generated indicate little or no degradation in the sample; alternatively, if there exists a relative molar abundance of the short amplicon compared to the longer amplicon, degradation of the nucleic acid in the sample can be assumed. This analysis is preferably done using multiple gene targets. This approach can further apply to the analysis of genomic DNA, where multiple amplicon targets (i.e., at least a short amplicon and a long amplicon) within a gene are designed, and the relative abundance of the amplicons following amplification from a genomic DNA sample is determined.

Nucleic Acid QC Metric

The invention provides methods for the assessment (e.g., quantification) of nucleic acid quality. This assessment of quality is termed the “quality metric” or “QC metric.” In some embodiments, the quality metric is an RNA quality metric. The value of this metric is not just in fully assessing FFPE-derived RNAs, but also permits broad use of the quality assay to assess all types of nucleic acid samples (e.g., RNA), including those from common biopsies or laser capture microdissection.

The relative differences in amplification efficiency associated with the different genes, regions amplified and the size of amplicon as the quality of the RNA progressively erodes provides the core information in arriving at the QC metric. From the observed trends in amplification efficiency, information is obtained with respect to the level of RNA integrity, variability, and usability e.g., in a microarray experiment. In some embodiments, the quality metric determination (a quality metric score or a relative quality metric comparison) guides the user in determining which samples can be successfully used in various downstream applications.

This quality metric information is key for not just predicting whether or not there is a good chance of success in downstream applications (e.g., PCR analysis, microarray analysis, cDNA library construction, northern analysis, southern blotting analysis) but also to provide an assessment of how good the data will be in accurately predicting what the true gene expression levels are. From the use of different sized amplicons from the same target gene, direct measure of the mean transcript length as calculated from the differences in amplification efficiency is determined. From the use of multiple amplicons across the length of the RNA transcripts, an assessment can be made of the relative availability and stability of the different parts of the transcript, and an assessment can be made of how well that nucleic acid sample will function in downstream analysis, e.g., data collection by the multiple probes on a gene chip, e.g., the Affymetrix® GeneChip®, especially given the GeneChip® probe bias toward the transcript 3′ ends in many of their chips. By analyzing the relative ratios of signal provided across multiple constitutive genes, an assessment can be made of variability in gene expression levels due to degradation of the RNA.

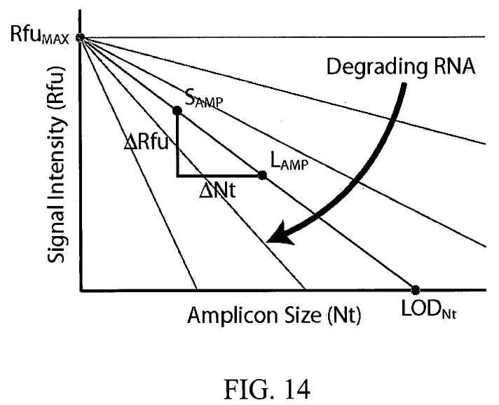

A primary approach in determining the RNA quality metric is to use the relative amplification efficiencies for each of the paired short (S_{AMP}) and long (L_{AMP}) amplicons as a prime measure of RNA integrity. Determination of the nucleic acid quality metric is shown schematically in FIG. 14. As seen in this figure, the different sized amplicons derived from the same gene demonstrate different absolute levels (reflecting different molar concentrations), reflective of the level of the degradation within a given sample. That is, the amplification efficiency of amplicons of increasingly larger size show decreasing signal intensity (e.g., Rfu) as the sample becomes degraded, reflecting the greater impact of degradation on the amplification of larger nucleic acids (e.g., the RNA transcripts). As seen in the figure, the differences in amplification efficiency as measured in relative fluorescence units (Rfu's) versus the differences in amplicon size in nucleotides (Nt) can be expressed as a slope, where:
slope=ΔRfu/ΔNt, wherein:
ΔRfu=Rfu(S_{AMP})−Rfu(L_{AMP}); S_{AMP}=small amplicon, L_{AMP}=large amplicon;
ΔNt=Nt(S_{AMP})−Nt(L_{AMP});
y-intercept=Rfu_{MAX}; and
x-intercept=LOD_{Nt}.

Of these values, the slope is most useful for assessing RNA degradation, although the intercept values can also be used. To determine the QC metric, RNA samples with known levels of degradation ared analyzed via PCR where different sized amplicons are used. From these PCR data, values for slopes are calculated for one, two, three or more gene locations for each of a plurality of genes (e.g., a plurality of genes selected from the list in Table 2, for example, at least 5 genes, 10 genes, 15 genes, 20 genes, 25 genes, or at least 30 or more genes) to generate a significant number of data points (e.g., 75 data points) for use in analyzing RNA quality. A basic QC metric can be determined using the slope data individually or collectively by using multiple slopes and calculating the median, the 75% value, the 90% value or the interquartile range of these slope values, which are more robust than the mean and/or standard deviation. The empirical distribution is also informative in understanding the diversity of observed levels of degradation throughout the population.

In some embodiments, trend analysis is performed to assess specific patterns of transcript degradation, and their correlation with likelihood of being successfully employed in downstream analysis (e.g., successfully generating good data via microarray analysis). Based on these more detailed analyses, a particular subset (or subsets) of information within the analysis data set that are sufficiently undegraded (e.g., sufficiently undegraded to permit microarray analysis) are identified. Thus, a predictive quality score can be generated where a score at or above that threshold is predictive for successful use in whatever particular applicant is desired, e.g., microarray analysis, and a score below that threshold will be predictive for unsuccessful analysis. Algorithms useful for generating the quality metric can also be readily derived.

In one embodiment, a nucleic acid quality metric can be described as follows. A total of six measurements are made on each of 24 different genes. The six measurements break down into measuring two different data points for three different regions of each gene. The two different data points for each region are linked in that they provide a measure of efficiency of amplification as a function of amplicon size. Specifically, the short amplicon (S_{AMP}), having a fixed and defined size in nucleotide length (Nt) provides a first amplification efficiency value, e.g., in relative fluorescence units (Rfu's) or other measure of amplification efficiency, and the long amplicon (L_{AMP}), having a fixed and defined size in nucleotide length that is longer than the S_{AMP}, provides a second amplification efficiency value, e.g., in Rfu's. The Rfu values are intrinsically linked to the size of the amplicon, as measured in nucleotides (Nt), and the degree of degradation on the RNA. Using these data, the slopes and intercepts are calculated and a QC metric value is determined.

Use of the nucleic acid quality metric also applies equally to genomic DNA. In these methods using genomic DNA, at least two primer pairs (for example, two primer pairs for generating a short and a long amplicon), and more preferably three primer pairs, are used to generate amplicons of varying lengths for specific sites on the DNA. The lengths of the amplicons is not an absolute; however, in preferred embodiments, the length of the shortest amplicon(s) is kept at a minimum (e.g., 40-60 base pairs) for the purpose of being able to detect degraded targets. The relative abundance of the short amplicon compared to the longer amplicon(s) provides a basis for observing, and in some embodiments quantitating, the amount of DNA degradation that is present, as in FIG. 14. As was the case in RNA, multiple sequence regions within the genomic DNA can be targeted for amplification where these regions can be monitored for degradation independently, e.g., as determined by the measurement of the slope or intercept, or can contribute to the cumulative measure of degradation as determined by calculating a mean or determining a trend, similar to those used for calculating RNA QC metrics.

Kits

The present invention provides articles of manufacture, for example, kits. A kit of the invention can include any assemblage components that are necessary or facilitate any method of the invention. The components of the kits of the invention are not particularly limited or restricted. Kits of the invention can optionally contain written instructions describing how to use the kit and/or conduct the methods of the invention.

The kits of the invention can provide any or all of the synthetic oligonucleotides used in methods described herein. For example, kits of the invention can include, but not limited to, primers suitable for reverse transcription and first strand and second strand cDNA synthesis, primers (e.g., any number of target primer pairs) directed to any gene, RNA or DNA of interest (for example, any of the reference genes of Table 2), paris of primers directed to any gene, RNA or DNA site of interest, universal primer(s) and/or semi-universal primer(s). In some embodiments, in the case where target primer pairs are provided, the 3′ end of the forward primer is not more than 20 base pairs from the 3′ end of the reverse primer when the target primers are hybridized to their cognate nucleic acid target. It is understood that the kits of the invention are not limited to primers specific for the genes provided in Tables 1 and 2, as the invention also provides guidance for the use of other probes directed to any other suitable genes.

Kits of the invention can include, but not limited to: instruments and/or containers for sample collection, apparatus and/or reagents for sample collection, apparatus and/or reagents for purification/isolation of RNA from any source, e.g., blood or FFPE samples, a reverse transcriptase, a thermostable DNA-dependent DNA polymerase suitable for use in PCR, and/or free deoxyribonucleotide triphosphates. In some embodiments, the enzyme comprising reverse transcriptase activity and thermostable DNA-dependent DNA polymerase activity are the same enzyme, e.g., Thermus sp. ZO5 polymerase or Thermus thermophilus polymerase.

Kits of the invention can also optionally comprise a container or plurality of containers to hold all of the components or any subset of components of the kit. Kits of the invention can be packaged for convenient storage and/or shipping. The components of the kits may be provided in one or more containers within the kit, and the components may be packaged in separate containers or may be combined in any fashion. In some embodiments, kits of the invention can provide materials to facilitate high-throughput analysis of multiple samples.

Kits of the invention can also optionally comprise software to assist in the analysis of data generated using the biochemical components of the kit. Software can include methods to design primers and multiplexes, methods and algorithms for the analysis of data generated including the calculation of nucleic acid QC metrics, determination and quantitation of degradation, and detection and quantitation of specific DNA and RNA sequences and molecules.

Integrated Systems

In some embodiments, the invention provides integrated systems for executing methods of the invention. For example, the invention provides systems for amplifying members of a population of degraded nucleic acids in a sample, systems for producing a gene expression profile from a degraded RNA sample, and systems for determining nucleic acid quality (i.e., for producing a nucleic acid quality metric) in a nucleic acid sample.

The systems can include instrumentation and means for interpreting and analyzing collected data, especially where the means for determining the nucleic acid QC metric or gene expression profile comprises algorithms and/or electronically stored information (e.g., collected fluorescence values, etc). Each part of an integrated system is functionally interconnected, and in some cases, physically connected. In some embodiments, the integrated system is automated, where there is no requirement for any manipulation of the sample or instrumentation by an operator following initiation of the analysis.

A system of the invention can include instrumentation. For example, the invention can include a detector such as a fluorescence detector (e.g., a fluorescence spectrophotometer). A detector or detectors can be used in conjunction with the invention, e.g., to monitor/measure fluorescence value). For example, a detector can be in the form of an integrated capillary electrophoresis apparatus, or multiwell plate reader to facilitate high-throughput capacity. In some embodiments, the integrated systems include a thermal cycling device, or thermocycler, for the purpose of controlling the temperature of a reaction, e.g., during the phases of an RT-PCR reaction.

A detector, e.g., a fluorescence spectrophotometer, can be connected to a computer for controlling the spectrophotometer operational parameters (e.g., wavelength of the excitation and/or wavelength of the detected emission) and/or for storage of data collected from the detector (e.g., fluorescence measurements during a melting curve analysis). The computer may also be operably connected to the thermal cycling device to control the temperature, timing, and/or rate of temperature change in the system. The integrated computer can also contain the “correlation module” where the data collected from the detector is analyzed and where the nucleic acid metric value or expression profile is derived electronically. In some embodiments, the correlation module comprises a computer program that collects the capillary electrophoresis fluorescence readings from the detector and furthermore produces the gene expression profile from a degraded RNA sample and/or derives a quality metric of the nucleic acid sample based on the fluorescence data.

A typical system of the invention can include one or more gene-specific chimeric primer pairs, one or more universal primers, a suitable detector (with or without an integrated thermal cycling instrument), a computer with a correlation module, and instruction (electronic or printed) for the system user. Typically, the system includes a detector that is configured to detect one or more signal outputs (where the signals correspond to PCR amplicons). In some embodiments, the system can further contain reagents used in the target amplification process. These reagents can include but are not limited to one or more of a DNA polymerase with RT activity, suitable buffers, stabilizing agents, dyes or stains, dNTPs, , etc. Kits can be supplied to operate in conjunction with one or more systems of the invention.

A wide variety of signal detection apparatus is available, including photo multiplier tubes, spectrophotometers, CCD arrays, scanning detectors, phototubes and photodiodes, microscope stations, galvo-scans, microfluidic nucleic acid amplification detection appliances and the like. The precise configuration of the detector will depend, in part, on the type of label used for amplicon generation/detection. Detectors that detect fluorescence, phosphorescence, radioactivity, pH, charge, absorbance, luminescence, temperature, magnetism or the like can be used. Typical detector embodiments include light (e.g., fluorescence) detectors or radioactivity detectors. For example, detection of a light emission (e.g., a fluorescence emission) or other probe label is indicative of the presence or absence of a marker allele. Fluorescent detection is commonly used for detection of amplified nucleic acids (however, upstream and/or downstream operations can also be performed on amplicons, which can involve other detection methods). In general, the detector detects one or more label (e.g., light) emission from a probe label. The detector(s) optionally monitors one or a plurality of signals from an amplification reaction.

System instructions that correlate a detected signal with a gene expression profile or a nucleic acid QC metric are also a feature of the invention. For example, the instructions can include at least one look-up table that includes a correlation between the detected signals and the nucleic acid metric. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector. As noted, in one typical embodiment, the system instructions include at least one look-up table that includes a correlation between the detected signals and the RNA metric. The instructions also typically include instructions providing a user interface with the system, e.g., to permit a user to view results of a sample analysis and to input parameters into the system.

The system typically includes components for storing or transmitting computer readable data detected by the methods of the present invention, e.g., in an automated system. The computer readable media can include cache, main, and storage memory and/or other electronic data storage components (hard drives, floppy drives, storage drives, etc.) for storage of computer code. Data representing a gene expression profile or the nucleic acid quality metric can also be electronically, optically or magnetically transmitted in a computer data signal embodied in a transmission medium over a network such as an intranet or internet or combinations thereof. The system can also or alternatively transmit data via wireless, IR, or other available transmission alternatives.

During operation, the system typically comprises the nucleic acid sample that is to be analyzed. In various aspects, the sample comprises RNA, polyA RNA, cRNA, total RNA, cDNA, amplified cDNA, or the like.

The phrase “system that correlates” in the context of this invention refers to a system in which data entering a computer corresponds to physical objects or processes or properties external to the computer, e.g., amplicon generation, and a process that, within a computer, causes a transformation of the input signals to different output signals, e.g., a gene expression profile or nucleic acid quality metric. In other words, the input data, e.g., the fluorescence readings following amplicon generation, is transformed to output data, e.g., the gene expression profile or nucleic acid quality metric. The process within the computer is a set of instructions, or “program,” by which amplicon signals are recognized by the integrated system and attributed to a gene expression profile or nucleic acid quality metric. In addition there are numerous programs for computing, e.g., C/C++, Delphi and/or Java programs for GUI interfaces, and productivity tools (e.g., Microsoft Excel and/or SigmaPlot) for charting or creating look up tables of nucleic acid quality metric data. Other useful software tools in the context of the integrated systems of the invention include statistical packages such as SAS, Genstat, Matlab, Mathematica, and S-Plus and genetic modeling packages such as QU-GENE. Furthermore, additional programming languages such as Visual Basic are also suitably employed in the integrated systems of the invention.

For example, gene expression profiles or nucleic acid quality metric can be recorded in a computer readable medium, thereby establishing a database. Any file or folder, whether custom-made or commercially available (e.g., from Oracle or Sybase) suitable for recording data in a computer readable medium can be acceptable as a database in the context of the invention. Data regarding gene expression profiles or nucleic acid quality metrics as described herein can similarly be recorded in a computer accessible database. Optionally, gene expression profiles or nucleic acid quality metrics can be obtained using an integrated system that automates one or more aspects of the assay (or assays) used to determine the gene expression profile or nucleic acid quality metric. In such a system, input data corresponding to amplicon detection can be relayed from a detector, e.g., an array, a scanner, a CCD, or other detection device directly to files in a computer readable medium accessible to the central processing unit. A set of system instructions (typically embodied in one or more programs) encoding the correlations between amplicon detection and the gene expression profile or nucleic acid quality metric can be then executed by the computational device.

Typically, the system also includes a user input device, such as a keyboard, a mouse, a touchscreen, or the like, for, e.g., selecting files, retrieving data, reviewing tables of amplicon detection values, etc., and an output device (e.g., a monitor, a printer, etc.) for viewing or recovering the product of the statistical analysis.

Thus, in one aspect, the invention provides an integrated system comprising a computer or computer readable medium comprising set of files and/or a database with at least one data set that corresponds to predetermined or experimental values. The system also includes a user interface allowing a user to selectively view one or more of these databases. In addition, standard text manipulation software such as word processing software (e.g., Microsoft Word™ or Corel WordPerfect™) and database or spreadsheet software (e.g., spreadsheet software such as Microsoft Excel™, Corel Quattro Pro™, or database programs such as Microsoft Access™ or Paradox™) can be used in conjunction with a user interface (e.g., a GUI in a standard operating system such as a Windows, Macintosh, Unix or Linux system) to manipulate strings of characters corresponding to the detected amplicons or other features of the database.

The systems optionally include components for sample manipulation, e.g., incorporating robotic devices. For example, a robotic liquid control armature for transferring solutions (e.g., samples) from a source to a destination, e.g., from a microtiter plate to an array substrate, is optionally operably linked to the digital computer (or to an additional computer in the integrated system). An input device for entering data to the digital computer to control high throughput liquid transfer by the robotic liquid control armature and, optionally, to control transfer by the armature to the solid support can be a feature of the integrated system. Many such automated robotic fluid handling systems are commercially available. For example, a variety of automated systems are available from Caliper Technologies (Hopkinton, Mass.), which utilize various Zymate systems, which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). As an alternative to conventional robotics, microfluidic systems for performing fluid handling and detection are now widely available, e.g., from Caliper Technologies Corp. (Hopkinton, Mass.) and Agilent Technologies (Palo Alto, Calif.).

Systems for generating gene expression profiles or nucleic acid quality metrics of the present invention can, thus, include a digital computer with one or more of high-throughput liquid control software, thermocycler control software, image analysis software for analyzing data from marker labels, data interpretation software, a robotic liquid control armature for transferring solutions from a source to a destination operably linked to the digital computer, an input device (e.g., a computer keyboard) for entering data to the digital computer to control high throughput liquid transfer by the robotic liquid control armature and, optionally, an image scanner for digitizing label signals from labeled probes. The image scanner interfaces with the image analysis software to provide a measurement of, e.g., amplicon intensity, where the label intensity measurement is interpreted by the data interpretation software to show whether, and to what degree, the amplicon is present. The data so derived is then correlated with a gene expression profile or a nucleic acid quality metric.

### EXAMPLES

The following examples are offered to illustrate, but not to limit the claimed invention. One of skill will recognize a variety of non-critical parameters that may be altered without departing from the scope of the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Toxicology Multiplex UPM-PCR Assay

The present Example describes a multiplex UPM-PCR using a 24-gene panel focused on a number of classical toxicological response endpoints following pharmaceutical treatment of cultured cells.

These gene expression endpoints used in the analysis include a number of different inducible cytochromes, as well as genes that report on oxidative stress, DNA damage, cell proliferation, apoptosis and a number of other important toxicology-related pathways. The gene set used in the toxicology panel are listed in Table 3.

TABLE 3
Toxicology Gene Panel

| Gene | GenBank Accession No. |
|---|---|
| Ro-CYP1A1 | NM_012540 |
| Ro-CYP4A1 | M57718 |
| Ro-CYP2B1 | U30327 |
| Ro-CYP3A1 | L24207 |
| Ro-CYP2E1 | NM_031543 |
| Ro-HO1 | J02722 |
| TSC22 | L25785 |
| NADPH CYP | M12516 |
| UGTB1 | XM_214015 |
| Ro-aldDH | AE001898 |
| RoNQO | NM_017000 |
| Ro-ApoA-IV | M00002 |
| Ro-p21 | U24174 |
| p53 | NM_030989 |
| Ro-gadd45 | L32591 |
| Ro-gadd153 | U36994 |
| Ro-COX-2 | S67722 |
| caspase-3 | NM_012922 |
| Ro-cyclinD1 | NM_171992 |
| Ro-PCNA | NM_022381 |
| Ro-CycloA | NM_017101 |
| Ro-betaActin | NM_031144 |
| Ro-GAPDH | NM_017008 |
| kanamycin (Kan) | reference |

This particular assay gene panel has been applied in a number of experimental programs including a study of multiple glitazones. Glitazones, also known as thiazolidinediones, are a class of drugs that improve the physiology of patients with type 2 diabetes by reducing insulin resistance, increasing insulin sensitivity, reducing serum triglyceride and free fatty acid levels, increasing serum HDL levels, and increasing glucose uptake. The effects of the thiazolidinediones are mediated by the activation of a peroxisome proliferator-activated receptor gamma (PPAR-γ); see Ribon et al., (1998) “Thiazolidinediones and insulin resistance: Peroxisome proliferators activated receptor g activation stimulates expression of the CAP gene,” PNAS 95:14751-14756.

The 24 gene panel for hepatotoxicity was used to analyze the gene expression levels of three different glitazones—pioglitazone, rosiglitazone and troglitazone. All three drugs went through full FDA approval, but Troglitazone (Rezulin) was subsequently removed from the market due to reports of severe idiosyncratic hepatocellular injury. In contrast, clinical studies with rosiglitazone (Avandia) and pioglitazone (Actos) reported no evidence of drug-induced hepatotoxicity.

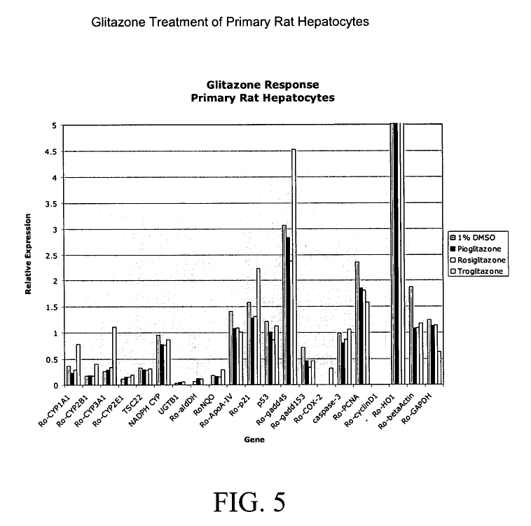

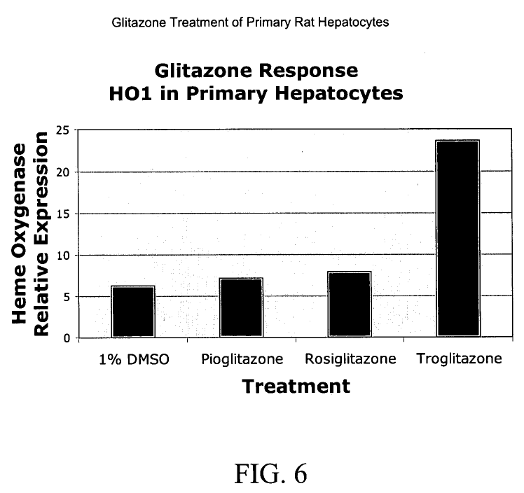

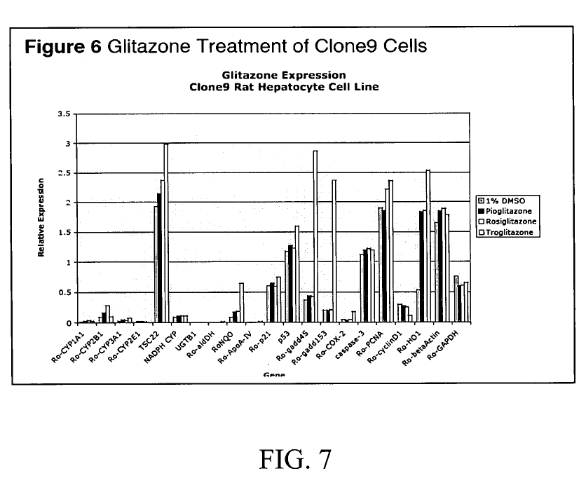

Representative data from the in vitro studies performed with the glitazone compounds using the Tox multiplex are shown in FIGS. 5-7, where treatments were performed in primary rat hepatocytes (FIGS. 5 and 6) and in the immortalized Clone9 hepatocyte cell line (FIG. 7). The study was performed to demonstrate that gene expression could have been used to differentiate the three compounds as significant differences were seen in a number of toxicologically relevant genes between pio- and rosi- and the more toxic troglitazone.

With respect to assay performance, the 24 gene toxicology multiplex, as seen in the glitazone study, demonstrates in a practical experimental setting much of the dynamic range of the assay. For example, in FIGS. 5 and 6, the expression values detected ranged from the low expressing UGBT1 (expression value of 0.032, 10.9% Cv) to the over expressed HO1 (expression value of 23.6, 13.6% Cv) representing nearly 3 logs of difference in expression intensity. The expression of Ro-HO1 expression is analyzed separately in FIG. 6 due to the different scales used on the vertical axis. Note that the Cv values include the biological variability of dosing the compounds on primary hepatocytes in 96-well culture in triplicate. Significant fold changes were also seen in FIG. 7, using the Clone9 hapatocyte cell line treatments. The differences in glitazone treatment were most pronounced in this cell line, versus the primary rat hepatocytes, e.g., a 7.6-fold induction of GADD45 and a 12-fold induction of GADD153.

### Example 2

### Tumor Tissue Multiplex UPM-PCR Assay

The present Example describes a multiplex UPM-PCR using a 33-gene panel that can differentiate four closely related tumor classes.

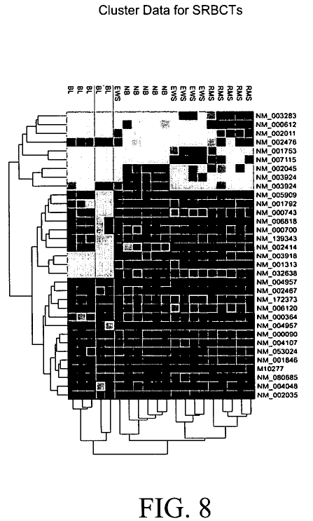

A study of multiplexed PCR assays for the differentiation and diagnosis of multiple forms of childhood cancer classified as small round blue-cell tumors (SRBCTs) was undertaken. SRBCTs represent four classes of tumor type: neuroblastoma, rhabdomyosarcoma, Burkitt's lymphoma and Ewing family tumors, that are important pediatric cancers. As the name eludes, SRBCTs are relatively difficult to differentiate in routine histology, but Khan et al., (“Classification and Diagnostic Prediction of Cancers Using Gene Expression Profiling and Artificial Neural Networks,” Nature 7:673-679 [2001]) found that significant differences can be seen in their gene expression patterns. In the 2001 study, a cDNA microarray with 6567 total genes was used to analyze 88 samples that included both tissues and cell lines for each of the four tumor types. Based on this microarray study, a 33 gene set was identified that is capable of differentiating the four SRBCT types. These genes plus four control genes were used to construct a 37-gene UPM-PCR multiplex. Preliminary data generated using this assay to analyze 20 tumor samples representing the different tumor types is shown in FIG. 8. Using basic hierarchical clustering to visualize the data, it is clear that the data from the multiplexed PCR assay will be capable of differentiating the different tumor types. The study is being extended by refining the gene list, and further, to analyze several hundred different SRBCT samples.

### Example 3

### Preparation of Test RNA Samples

Proper controls are a key feature in any scientific study. The present Example describes preparation of test control degraded RNA samples containing various degrees of RNA degradation for the purpose of developing and testing the methods of the invention. With these samples it is possible to directly compare the levels of gene expression and the impact of RNA degradation on these expression levels. This approach allows the synthetic creation of a broad range of degradation, as well as different types of mechanistic degradation, so that many levels and/or types of degradation can be studied.

Two types of human test RNAs are prepared, each in multiple ways to represent progressively greater levels of degradation. Type 1 is total RNAs derived from several different tissue types, including tissue mixes, that is degraded via chemical and enzymatic titrations. Type 2 is RNAs derived from fresh frozen and FFPE blocks all prepared from the same prostate tissue. The prostate tissue blocks are sliced and have RNAs prepared at specific time intervals. These FFPE samples are also placed under several different storage conditions reflective of common practices for FFPE sample storage.

Type 1 RNA Controls

Several different sources of RNA are used to generate the Type 1 RNA controls. The first source is RNA isolated from fresh frozen tissue, e.g. a tissue representative of the types of tissues to be analyzed in FFPE blocks such as prostate cancer tissue. These tissue samples are partitioned so that a portion of each is isolated for the preparation of Type 1 RNAs and the remainder used to generate FFPE tissue blocks as part of the Type 2 RNA controls.

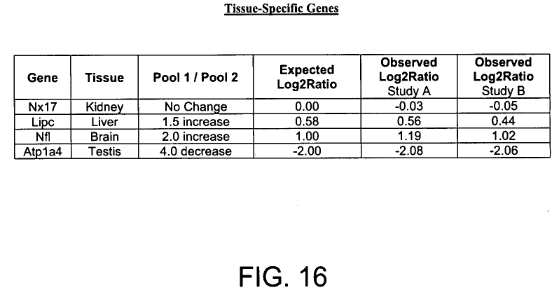

Titrated tissue pools are the second source of RNA for the preparation of Type I RNA controls. A system to evaluate microarray performance that utilized two pools of rat RNA has recently been developed (Rosenzweig et al., (2004) “Formulation of RNA Performance Standards for Regulatory Toxicogenomic Studies,” Society of Toxicology Annual Meeting, Abstract ID 1705, The Toxicologist CD, Volume 78:1-S). The pools are prepared by combining different amounts of RNA from four rat tissues: brain, kidney, liver and testicles (FIG. 11). Replicate targets of the two pools are prepared and hybridized. The expression results are compared based on their ability to detect known differences in expression at tissue specific genes. For example, if pool 1 contains 40% brain tissue and pool 2 contains 20% brain tissue, then a 2-fold change of expression should be detected in brain specific transcripts. These samples have been utilized in this system in preliminary studies. Typical results for four genes in two identical studies are shown in FIG. 16.

Approximately 200 “invariant” genes that are expressed in only one of the input tissues have been previously identified. The identification of the subset of invariant rat genes that are also tissue-specific in human is currently in progress. A set of 8 of these genes is selected and used in the assay. Two to four different tissue titrations are prepared and prostate tissue is included in the mixture instead of testis.

Once the full set of RNAs has been selected, they are used to generate the progressively degraded test RNAs. Titrations are performed using both enzymatic and chemical (e.g., NaOH treatment) degradation techniques. Enzymatic degradations are performed using two different types of ribonuclease, for example, RNase ONE™ Ribonuclease (Promega™ Corp., Madison, Wis.; see Promega Notes Magazine Number 38, August 1992, p. 1) and RNase III Ribonuclease (Ambion®, Inc., Austin, Tex.). RNase ONE™ is an engineered ribonuclease that carries no base specificity but is selective for single stranded RNA over double stranded structures. RNase III degrades double stranded RNA. The two enzymes are used individually and together to generate different patterns of RNA degradation. Classic base treatment using NaOH is the third RNA degradation method used. Base treatment is relatively indiscriminant and mimic some of the mechanisms of degradation related to the fixation process. The level of RNA degradation is initially tracked using different methods such as, for example, by electrophoresis using the Agilent Bioanalyzer, to assure that degradation is progressing. The electropherograms is then compared against those derived from FFPE samples to assure that the range of degradation present in these samples encompasses the degradation in the FFPE samples.

Type 2 RNA Controls

The Type 2 RNA controls are formalin fixed, paraffin embedded samples and their associated fresh frozen RNAs. In fact, the same tissue sources used for the prostate samples in the Type 1 RNAs is used for preparing tissue blocks. Therefore, a direct comparison between the RNAs generated in enzymatic and base treatments and those generated from FFPE tissue blocks can be made.

An important issue associated with existing FFPE tissue blocks is storage. To assess the impact of storage on RNA and DNA degradation, the tissue blocks prepared in this study are stored under several different conditions and periodically are cut to have RNA isolated from them. From each block, RNA is isolated at one day, and 1, 3, 6, 12, 18 and 24 months. Efforts are made to assure that each tissue sampling represents a similar mix of tissue structure and cell types. The storage conditions are −20° C., 4° C., room temperature, and 37° C. The different storage conditions provide a significant range of degradation rates over the 2 year duration for which they are stored. Upon isolation all Type 1 and Type 2 RNAs for this study are immediately aliquoted and then stored at −80° C. at a relatively high concentration to assure maximum stability.

### Example 4

### Tumor Metasisis Multiplex PPM-PCR Assay

The present Example describes a multiplex PPM-PCR using an 18-gene panel analyzing gene expression in normal and cancerous breast tissue. This PPM-PCR analysis illustrates a large number of analyzed transcripts while still limiting the amount of target sequence used to less that 80 nucleotides. The genes used in the analysis are well documented genes known for their differential response in metastatic cells across a broad range of tissue and tumor types. The genes used in the multiplex are listed in Table 4 below. This table indicates the amount of target sequence that is amplified and their final amplicon size using the PPM-PCR method.

TABLE 4

| | | Total | Target-Specific |
|---|---|---|---|
| | GenBank | Amplicon Size | Sequence Amplified |
| Gene | Accession Number | (base pairs) | (base pairs) |
| P21 | NM_000389 | 99 | 40 |
| VEGF | BC065522 | 103 | 41 |
| PLAT | NM_000930 | 107 | 44 |
| CCNE2 | NM_004702 | 111 | 46 |
| GapDH | NM_002046 | 115 | 48 |
| CycA | BC000689 | 121 | 50 |
| MP1 | NM_014889 | 125 | 52 |
| MUC-1 | NM_002456 | 129 | 54 |
| cynD1 | BC001501 | 133 | 56 |
| SOX-9 | NM_000346 | 137 | 58 |
| Srvin | AB028869 | 142 | 60 |
| EGF | NM_00522 | 145 | 62 |
| P53 | AF307851 | 149 | 64 |
| MYC | NM_002467 | 153 | 66 |
| MMP9 | NM_004994 | 157 | 68 |
| TFRC | NM_003234 | 161 | 70 |
| ERBB2 | NM_004448 | 165 | 72 |
| Actin | NM_00101 | 169 | 74 |
| bFGF2 | J04513 | 177 | 80 |

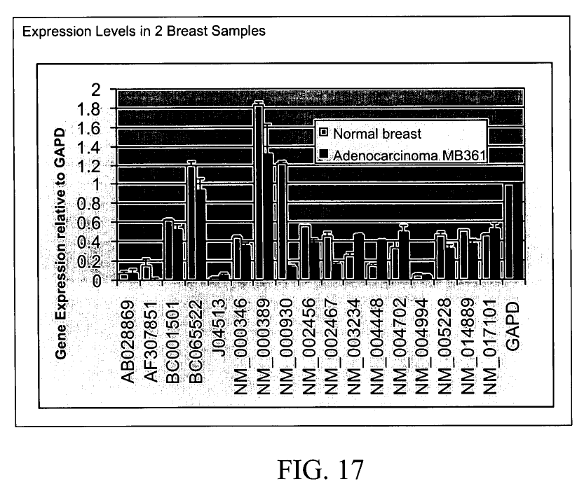

The electophoresis trace following the multiplex reaction is shown in FIG. 17. All 19 genes analyzed show good signal. The analysis of normal breast tissue and an adenocarcinoma derived cell line are shown. The reactions were run in triplicate with the error bars shown.

### Example 5

### Comparison Analysis using Proximal-Primer Multiplexed PCR (PPM-PCR) with FFPE Samples

The present Example describes a comparative analysis of UMP-PCR and PPM-PCR methods using an intact tissue-derived RNA sample and a FFPE-derived RNA sample.

Studies using the PPM-PCR method in the analysis of FFPE-derived RNA samples were conducted. Two different multiplexes were developed that target a set of human reference genes. The first multiplex utilized the universal primed multiplexed PCR methods (UPM-PCR) to create a 24-gene reference multiplex. The average size of amplicons for this multiplex was 250 base pairs. Subtracting the universal primer tails, the average size of target-specific sequence amplified from the mRNA was 210 base pairs. The second multiplex utilized the PPM-PCR method that targeted between 40-60 base pairs of mRNA-specific sequence (with a mean of 50 base pairs), but through the use of universal primers and spacer sequences to generate amplicon products between 80 and 150 total base pairs in length. The second multiplex using the PPM-PCR method targeted ten different human reference genes.

The first RNA sample was derived from a FFPE human prostate block. The age of the block was 14 years, stored at room temperature. The second sample was human universal reference total RNA (Clontech Laboratories, Inc., Mountain View, Calif.; Catalog No. 636538). In the analysis, 20 ng of RNA from each sample was run as multiple replicates using the two different multiplexes and then analyzed on a Beckman-Coulter™ CEQ™ 8800 Genetic Analysis System.

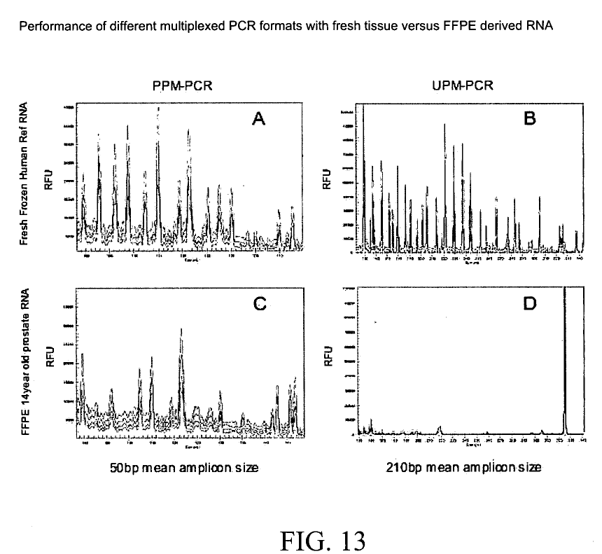

A direct comparison of the two methods using the two different RNA samples was performed. The data from this analysis is shown in FIG. 13. From the data it is immediately apparent that the size of the amplicon strongly impacts the ability to detect signal from the FFPE sample. The larger amplicon UPM-PCR method using the intact reference RNA sample generated strong amplicon signals (FIG. 13B), but that same method using the degraded FFPE sample generated very little amplicon data (FIG. 13D). In contrast, the PPM-PCR method utilizing smaller amplicons generated strong amplification signals in both the undegraded reference RNA sample (FIG. 13A) as well as the degraded FFPE RNA sample (FIG. 13C). The PPM-PCR method generated a full complement of gene data with about 25% of the signal intensity as compared to the undegraded universal reference RNA (FIG. 13A). Note that the relative gene ratios cannot be directly compared since the samples represented RNAs from different tissues of origin. The large peak in FIG. 13D is form spiked control transcript.

It is understood that the examples and embodiments described herein are for illustrative purposes only and that various modifications or changes in light thereof are suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method for amplifying members of a population of degraded nucleic acids in a sample, the method comprising:
a) providing a sample comprising said population of degraded nucleic acids;
b) providing target primer pairs, wherein
i) each target primer pair comprises a forward target primer and a reverse target primer;
ii) said forward and reverse target primers each comprise (A) a target-specific nucleotide sequence that is complementary to a nucleotide subsequence of at least one member of the population of degraded nucleic acids, and (B) at least one universal priming sequence, wherein the universal priming sequence is 5′ relative to the target-specific sequence; and
c) annealing said target primer pairs to their cognate degraded nucleic acid;
d) enzymatically producing a plurality of products corresponding to subsequences of said cognate degraded nucleic acids;
e) enzymatically amplifying said plurality of products using at least one universal primer to produce a plurality of target amplicons, wherein said universal primer comprises nucleotide sequence that is complementary to said universal priming sequence, thereby amplifying members of a population of degraded nucleic acids.

2. The method of claim 1, wherein said nucleic acids are DNA, and said population of degraded nucleic acids in said sample has a mean size of not more than 1,000 nucleotides.

3. The method of claim 1, wherein said nucleic acids are RNA, and said population of degraded nucleic acids in said sample has a mean size of not more than 600 nucleotides.

4. The method of claim 1, wherein said nucleic acids are RNA, and said population of degraded nucleic acids in said sample has a mean size of about or less than 450 nucleotides.

5. The method of claim 1, wherein said nucleic acids are RNA, and said population of degraded nucleic acids in said sample has a mean size of about or less than 300 nucleotides.

6. The method of claim 1, wherein said members of a population of degraded nucleic acids comprises between about 2 and about 100 members.

7. The method of claim 1, wherein said members of a population of degraded nucleic acids comprises between about 10 and about 40 members.

8. The method of claim 1, wherein said members of a population of degraded nucleic acids correspond to expressed gene nucleotide sequences.

9. The method of claim 8, wherein at least one expressed gene nucleotide sequence is a constitutively-expressed reference gene sequence.

10. The method of claim 8, further comprising comparing a level of expression of at least one expressed gene sequence to a level of expression of at least one other expressed gene sequence.

11. The method of claim 8, wherein said expressed gene nucleotide sequences comprise tissue-specific gene sequences.

12. The method of claim 1, wherein said population of degraded nucleic acids comprises RNA or DNA.

13. The method of claim 1, wherein said population of degraded nucleic acids comprises RNA, and the step of producing a plurality of products comprises reverse transcription.

14. The method of claim 1, wherein said sample comprises mammalian RNA comprising 28S and 18S RNA species, and the quantitative ratio of 28S RNA to 18S RNA is not more than 2.0:1.

15. The method of claim 1, wherein said sample comprises mammalian RNA comprising 28S and 18S RNA species, and the quantitative ratio of 28S RNA to 18S RNA is not more than 1.8:1.

16. The method of claim 1, wherein said sample comprises total cellular RNA.

17. The method of claim 1, wherein said sample comprises polyadenylated RNA.

18. The method of claim 1, wherein said sample is derived from a tissue sample that has undergone fixation.

19. The method of claim 1, wherein said sample is derived from a paraffin-embedded tissue sample.

20. The method of claim 1, wherein at least one member in the plurality of products comprises not more than 200 base pairs of nucleotide sequence corresponding to said degraded nucleic acids.

21. The method of claim 1, wherein at least one member in the plurality of products comprises not more than 100 base pairs of nucleotide sequence corresponding to said degraded nucleic acids.

22. The method of claim 1, wherein at least one member in the plurality of products comprises not more than 80 base pairs of nucleotide sequence corresponding to said degraded nucleic acids.

23. The method of claim 1, wherein at least one member in the plurality of products comprises not more than 60 base pairs of nucleotide sequence corresponding to said degraded nucleic acids.

24. The method of claim 1, wherein said enzymatically producing a plurality of products comprises enzymatic nucleic acid polymerization by the polymerase chain reaction (PCR).

25. The method of claim 24, wherein said PCR is a multiplex PCR.

26. The method of claim 25, wherein said multiplex PCR employs between about 2 and about 100 target primer pairs.

27. The method of claim 25, wherein said multiplex PCR employs between about 10 and about 40 target primer pairs.

28. The method of claim 1, wherein at least one target primer further comprises at least one spacer nucleotide between said target-specific sequence and said universal priming sequence.

29. The method of claim 1, wherein the concentration of each target primer in the target primer pair is less than the concentration of said at least one universal primer.

30. The method of claim 1, wherein the ratio of the concentration of each target primer pair to the concentration of the universal primer is between about 1:2 and 1: 1000.

31. The method of claim 1, wherein the ratio of the concentration of each target primer pair to the concentration of the universal primer is between about 1:10 and 1:100.

32. The method of claim 1, wherein enzymatically amplifying said plurality of products comprises enzymatic nucleic acid polymerization by the polymerase chain reaction (PCR).

33. The method of claim 1, wherein the length of one target amplicon is different than the length of at least a second target amplicon.

34. The method of claim 1, wherein each primer in a target primer pair comprises a 3′ end, and wherein said 3′ end of said forward primer is not more than 20 base pairs from said 3′ end of said reverse primer when said target primers are hybridized to a cognate nucleic acid target.

35. The method of claim 1, wherein at least one target amplicon comprises a label.

36. The method of claim 1, wherein a plurality of said target amplicons each comprise a different label.

37. The method of claim 1, comprising detecting said plurality of target amplicons.

38. The method of claim 37, wherein said detecting step is by capillary electrophoresis analysis.

39. The method of claim 37, wherein said detecting step is by hybridization analysis.

40. The method of claim 39, wherein said hybridization analysis comprises an array hybridization.

41. The method of claim 39, wherein said hybridization analysis comprises a bead system hybridization.

42. A method for determining nucleic acid quality in a nucleic acid sample, the method comprising:
a) providing a nucleic acid sample;
b) providing at least two target primer pairs, wherein:
i) each target primer pair comprises a forward target primer and a reverse target primer;
ii) said forward and reverse target primers each comprise a target-specific nucleotide sequence that is complementary to a subsequence of at least one nucleic acid in said sample; and
c) annealing said target primer pairs to their cognate nucleic acid;
d) enzymatically producing at least two products corresponding to nucleotide subsequences of said cognate nucleic acid, wherein each nucleotide subsequence is a different length;
e) enzymatically amplifying said products to produce at least two target amplicons;
f) quantitating said at least two target amplicons; and
g) comparing quantities of said target amplicons, thereby determining nucleic acid quality in said sample.

43. The method of claim 42, wherein said at least two target primer pairs each anneal to the same nucleic acid.

44. The method of claim 43, wherein said at least two products comprise overlapping nucleotide subsequences.

45. The method of claim 43, wherein said at least two products do not comprise overlapping nucleotide subsequences.

46. The method of claim 42, wherein said forward and reverse target primers each further comprise at least one universal priming sequence, wherein said universal priming sequence is 5′ relative to the target-specific sequence; and wherein enzymatically amplifying said products comprises incorporating at least one universal primer to produce at least two target amplicons, wherein said universal primer comprises nucleotide sequence that is complementary to said universal priming sequence.

47. The method of claim 45, wherein at least one target primer further comprises at least one spacer nucleotide between said target-specific sequence and said universal priming sequence.

48. The method of claim 42, wherein said at least two target primer pairs anneal to different nucleic acids.

49. The method of claim 42, wherein each product comprises nucleotide sequence corresponding to a cognate nucleic acid, where the nucleotide sequence has a size range selected from about (i) 40-60 base pairs, inclusive; (ii) 100-120 base pairs, inclusive; and (iii) 180-200 base pairs, inclusive, where the nucleotide sequence size range for one product is different than the nucleotide sequence size range of any other product.

50. The method of claim 42, wherein said at least two target primer pairs consists of two target primer pairs.

51. The method of claim 42, wherein said at least two target primer pairs consists of three target primer pairs.

52. The method of claim 51, wherein each product comprises nucleotide sequence corresponding to a cognate nucleic acid, where the nucleotide sequence has a size range selected from about (i) 40-60 base pairs, inclusive; (ii) 100-120 base pairs, inclusive; and (iii) 180-200 base pairs, inclusive, where the nucleotide sequence size range for one product is different than the nucleotide sequence size range of the remaining two products.

53. The method of claim 42, wherein said comparing quantities of said target amplicons comprises comparing relative molar concentrations of said target amplicons.

54. The method of claim 53, wherein the relative molar concentration of one target amplicon is less than the relative molar concentration of at least a second target amplicon, thereby indicating degraded nucleic acid.

55. A method for producing a gene expression profile from a degraded RNA sample, the method comprising the steps:
a) providing a sample comprising degraded RNA, wherein said RNA corresponds to expressed genes;
b) providing a plurality of target primer pairs, wherein:
i) each target primer pair comprises a forward target primer and a reverse target primer;
ii) said forward and reverse target primers each comprise (A) a target-specific nucleotide sequence that is complementary to a nucleotide subsequence of at least one degraded RNA in said sample, and (B) at least one universal priming sequence, wherein the universal priming sequence is 5′ relative to the target-specific sequence; and
c) annealing said target primer pairs to their cognate degraded RNA;
d) enzymatically producing a plurality of products corresponding to subsequences of said cognate degraded RNA;
e) enzymatically amplifying said plurality of products using at least one universal primer to produce a plurality of target amplicons, wherein said universal primer comprises nucleotide sequence that is complementary to said universal priming sequence, thereby producing a gene expression profile from a degraded RNA sample.

56. The method of claim 55, wherein said plurality of target primer pairs comprises between about between about 2 and about 100 target primer pairs.

57. The method of claim 55, wherein said plurality of target primer pairs comprises between about between about 10 and about 40 target primer pairs.

58. The method of claim 55, wherein at least one target amplicon comprises a label.

59. The method of claim 55, wherein a plurality of said target amplicons each comprise a different label.

60. The method of claim 55, comprising detecting said plurality of target amplicons.

61. The method of claim 55, wherein said detecting step is by capillary electrophoresis analysis.

62. The method of claim 55, wherein said detecting step is by hybridization analysis.

63. The method of claim 55, wherein said hybridization analysis comprises an array hybridization.

64. The method of claim 55, wherein said hybridization analysis comprises a bead system hybridization.

65. A kit for analyzing a sample, said sample comprising degraded nucleic acid, the kit comprising:
a) a plurality of target primer pairs, wherein:
i) each target primer pair comprises a forward target primer and a reverse target primer;
ii) said forward and reverse target primers each comprise (A) a target-specific nucleotide sequence that is complementary to a nucleotide subsequence of at least one degraded nucleic acid in said sample, and (B) at least one universal priming sequence, wherein the universal priming sequence is 5′ relative to the target-specific sequence; and
iii) each primer in a target primer pair comprises a 3′ end, and wherein said 3′ end of said forward primer is not more than 20 base pairs from said 3′ end of said reverse primer when said target primers are hybridized to a cognate nucleic acid target; and
b) instructions for analyzing a sample comprising degraded nucleic acids.
